SEQUENCE LISTING (8 sequences)

SEQ ID NO: 1 - DNA, 1770 bp, synthetic construct
  source 1..1770: mol_type = other DNA
aggggtgtgtttcgccgagaagcacacaagagtgagatcgcccatcggtataatgatttgggagaacaacatttcaaaggcctagtcctgattgccttttcccagtatctccagaaatgctcatacgatgagcatgccaaattagtgcaggaagtaacagactttgcaaagacgtgtgttgccgatgagtctgccgccaactgtgacaaatcccttcacactctttttggagataagttgtgtgccattccaaacctccgtgaaaactatggtgaactggctgactgctgtacaaaacaagagcccgaaagaaacgaatgtttcctgcaacacaaagatgacaaccccagcctgccaccatttgaaaggccagaggctgaggccatgtgcacctcctttaaggaaaacccaaccacctttatgggacactatttgcatgaagttgccagaagacatccttatttctatgccccagaacttctttactatgctgagcagtacaatgagattctgacccagtgttgtgcagaggctgacaaggaaagctgcctgaccccgaagcttgatggtgtgaaggagaaagcattggtctcatctgtccgtcagagaatgaagtgctccagtatgcagaagtttggagagagagcttttaaagcatgggcagtagctcgtctgagccagacattccccaatgctgactttgcagaaatcaccaaattggcaacagacctgaccaaagtcaacaaggagtgctgccatggtgacctgctggaatgcgcagatgacagggcggaacttgccaagtacatgtgtgaaaaccaggcgactatctccagcaaactgcagacttgctgcgataaaccactgttgaagaaagcccactgtcttagtgaggtggagcatgacaccatgcctgctgatctgcctgccattgctgctgattttgttgaggaccaggaagtgtgcaagaactatgctgaggccaaggatgtcttcctgggcacgttcttgtatgaatattcaagaagacaccctgattactctgtatccctgttgctgagacttgctaagaaatatgaagccactctggaaaagtgctgcgctgaagccaatcctcccgcatgctacggcacagtgcttgctgaatttcagcctcttgtagaagagcctaagaacttggtcaaaaccaactgtgatctttacgagaagcttggagaatatggattccaaaatgccattctagttcgctacacccagaaagcacctcaggtgtcaaccccaactctcgtggaggctgcaagaaacctaggaagagtgggcaccaagtgttgtacacttcctgaagatcagagactgccttgtgtggaagactatctgtctgcaatcctgaaccgtgtgtgtctgctgcatgagaagaccccagtgagtgagcatgttaccaagtgctgtagtggatccctggtggaaaggcggccatgcttctctgctctgacagttgatgaaacatatgtccccaaagagtttaaagctgagaccttcaccttccactctgatatctgcacacttccagagaaggagaagcagattaagaaacaaacggctcttgctgagctggtgaagcacaagcccaaggctacagcggagcaactgaagactgtcatggatgactttgcacagttcctggatacatgttgcaaggctgctgacaaggacacctgcttctcgactgagggtccaaaccttgtcactagatgcaaagacgccttagcc

SEQ ID NO: 2 - DNA, 819 bp, synthetic construct
  source 1..819: mol_type = other DNA
gaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcct

SEQ ID NO: 3 - DNA, 1755 bp, synthetic construct
  source 1..1755: mol_type = other DNA
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgtagctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtcagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaaatgtgccagtctccaaaaatttggagaaagagctttcaaagcatgggcagtggctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaactgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 4 - DNA, 831 bp, synthetic construct
  source 1..831: mol_type = other DNA
caagtggacaccacaaaggcagtgatcactttgcagcctccatgggtcagcgtgttccaagaggaaaccgtaaccttgcattgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcat

SEQ ID NO: 5 - DNA, 30 bp, synthetic construct
  source 1..30: mol_type = other DNA
ggtggtggtggttctgaagcacacaagagt

SEQ ID NO: 6 - DNA, 30 bp, synthetic construct
  source 1..30: mol_type = other DNA
gactctagaggctaaggcgtctttgcatct

SEQ ID NO: 7 - DNA, 37 bp, synthetic construct
  source 1..37: mol_type = other DNA
gcctcgagaaaagagaagtggttaatgccaccaaggc

SEQ ID NO: 8 - DNA, 29 bp, synthetic construct
  source 1..29: mol_type = other DNA
acagaaccaccaccaccaggagctgatga

(19) United States
(12) Patent Application Publication
(10) Pub. No.: US 2024/0083976 A1
(43) Pub. Date: Mar. 14, 2024

(54) SERUM ALBUMIN-BASED FUSION PROTEIN, AND NANO-ASSEMBLY, PREPARATION METHOD THEREFOR AND APPLICATION THEREOF

(71) Applicant: SOUTH CHINA UNIVERSITY OF TECHNOLOGY, Guangdong (CN)

(72) Inventors: Jun WANG, Guangdong (CN); Yanan FAN, Guangdong (CN); Song SHEN, Guangdong (CN); Qianni YE, Guangdong (CN)

(73) Assignee: SOUTH CHINA UNIVERSITY OF TECHNOLOGY, Guangdong (CN)

(21) Appl. No.: 18/363,747

(22) Filed: Aug. 2, 2023

Related U.S. Application Data
(63) Continuation of application No. PCT/CN22/74079, filed on Jan. 26, 2022.

(30) Foreign Application Priority Data
Feb. 5, 2021 (CN) 202110164375.1

(51) Int. Cl. C07K 14/76; A61K 47/64; A61K 47/68; A61K 47/69; C07K 14/735; C07K 19/00

(52) U.S. Cl. CPC C07K 14/76; A61K 47/643; A61K 47/6849; A61K 47/6937; C07K 14/70535; C07K 19/00; C07K 2319/31

(57) ABSTRACT

Provided is a fusion protein, comprising a protein having a hydrophobic region, a peptide linker, a protein fusion receptor. The peptide linker links the protein having a hydrophobic region to the protein fusion receptor. The protein fusion receptor is an Fc receptor fragment that specifically recognizes an Fc fragment of an antibody, and the protein having a hydrophobic region is a serum albumin. Also provided is a nano-assembly consisting of the fusion protein and a hydrophobic degradable polyester and a derivative thereof. Also provided is an application of the nano-assembly having excellent stability in an antibody delivery platform. The constructed nano-assembly platform is creatively applied in the preparation of immunotherapeutic drugs or therapeutic drugs for tumors or autoimmune diseases or inflammations.

### CROSS-REFERENCE TO RELATED APPLICATION

This application is a continuation of international application of PCT application serial no. PCT/CN2022/074079 filed on Jan. 26, 2022, which claims the priority benefit of China application no. 202110164375.1 filed on Feb. 5, 2021. The entirety of each of the above mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Jul. 21, 2023, is named 136166US-SEQUENCING_LIST and is 13,472 bytes in size.

### TECHNICAL FIELD

The present disclosure relates to the technical field of pharmaceutical technology, in particular to a serum albumin-based fusion protein, a nano-assembly, and the preparation method and applications thereof.

### RELATED ART

CTLA-4, PD-1, PD-L1 and other immune checkpoint blocking antibodies have been approved for the treatment of various types of tumors, and have achieved phased results. However, with the extensive and in-depth research, a large number of clinical experimental results have shown that immune checkpoint blocking and other immunotherapies have significant differences in treatment efficacy in different types of tumors and the same type of tumors of different patients, and the overall clinical response rate is low. Many monoclonal antibody drugs have repeatedly failed in clinical applications, and there is an urgent need to develop new strategies to improve the anti-tumor effect of antibody drugs.

Antibody co-administration or the preparation of dual/multi specific antibodies through genetic engineering are used to overcome the problem of insufficient potency of monoclonal antibody drugs. At present, researchers have developed over 100 bispecific antibody construction models, and over 85 bispecific antibodies are currently in clinical development stage. Although bispecific/multi-specific antibodies can significantly improve their titer and disease treatment efficacy through dual or multiple recognition, their structural design complexity is high, and the complexity of design, preparation, purification, and other processes increases significantly compared to monoclonal antibodies. Moreover, most of them are prepared by chemical coupling and DNA recombination technology, which requires chemical modification on the effective monoclonal antibody, which will inevitably affect the antigen binding ability of the antibody itself. At the same time, there are also shortcomings such as short half-life, complex administration mode, poor stability, poor solubility and high cost. At present, no bispecific/multi-specific antibody has been approved for the treatment of solid tumors. Therefore, if the design concept of bispecific/multi-specific antibodies can be used to develop new and convenient strategies to achieve “multivalent”, “multi-specific”, and “multifunctional” of monoclonal antibodies, it is expected to significantly improve the clinical efficacy of monoclonal antibodies and apply more monoclonal antibodies in development or clinical practice to the treatment of solid tumors.

Fixing multiple monoclonal antibodies on the surface of nanocarriers can simulate the functions of bispecific/multi-specific antibodies, achieving “multivalent”, “multi-specific”, and “multifunctional” of monoclonal antibodies. For example, the research group of Professor Jonathan P. Schneck of Johns Hopkins University in the United States built a nanoparticle with “double targeting” function by bonding the blocking PD-L1 monoclonal antibody and the activating 4-1BB monoclonal antibody to the surface of dextran iron particles at the same time. The nanoparticle could not only block the PD-L1/PD-1 inhibitory signal pathway, but also activate the 4-1BBL/4-1BB pathway. After intratumoral administration, the ability of cytotoxic T cells to kill tumor cells was significantly enhanced. It is a highly promising strategy for improving antibody efficacy by attaching multiple monoclonal antibodies to the surface of nanocarriers. However, the reported methods of immobilizing antibodies mainly utilize amino, carboxyl, and thiol groups of antibody molecules to bond them to the surface of particles, which has many problems. Firstly, the high molecular weight of antibodies and nanoparticles often leads to low reaction efficiency and difficult quality control; secondly, using the thiol groups generated by reduction or the rich amino groups on the surface of the antibody to react with particles not only complicates the reaction and purification process, but also destroys the advanced structure of the antibody or blocks the antigen recognition region of the therapeutic antibody, significantly reducing the antibody's ability to recognize antigens; moreover, currently reported carriers for antibody delivery are mostly polystyrene nanoparticles, iron oxide nanoparticles, etc., which have poor biocompatibility. These greatly hinder the clinical conversion of “nano antibodies” based on carrier systems.

It is expected to significantly improve the anti-tumor effect of existing monoclonal drugs by constructing antibody delivery carriers with clinical conversion prospects, developing convenient, efficient, and controllable antibody drug binding methods, solving the problems of low reaction efficiency and complex process of existing nano carrier fixed antibody methods, and achieving “multivalence”, “multi specificity”, and “multifunctional” of antibody drugs.

There are multiple Fc receptors on the surface of monocytes such as macrophages, among which FcγRI can recognize and bind Fc fragments of antibodies with specificity and high affinity. It does not involve complex chemical reactions to binding with monoclonal antibody drugs through FcγRI and has almost no impact on the structure and function of antibody drugs.

Human serum albumin is a protein with 585 amino acids, which is an important part of maintaining osmotic pressure in serum, and plays a role of carrier in transporting endogenous and exogenous substances. We have noticed that albumin has seven binding sites with long-chain fatty acid, and the binding sites are relatively open. Its hydrophobic cavity combines with the carboxylic acid portion of lipids through arginine or lysine residues together with tyrosine or serine by hydrogen bonding and electrostatic interactions. So, based on the research and development of the antibody delivery platform in the early stage, we innovatively proposed to integrate FcγRI and albumin fuse to form a recombinant protein, and then use albumin and hydrophobic polylactic acid polymer materials to construct nanoparticles. With FcγRI existing on the surface of the particles recognizes and combines therapeutic monoclonal antibody drugs, we construct novel dual/multi specific antibodies for the treatment of tumors, immune related diseases, and other related diseases.

### SUMMARY OF INVENTION

Based on the above, the purpose of the present disclosure is to provide a fusion protein, which can be used to deliver at least one antibody.

The specific technical solutions are as follows:

A fusion protein for delivering at least one antibody, including serum albumin and a protein receptor, wherein the serum albumin is connected to the protein receptor directly or through a peptide connector; the protein receptor is an Fc receptor.

The second purpose of the present disclosure is to provide a nano-assembly for delivering at least one antibody.

A nano-assembly for delivering at least one antibody, wherein the nano-assembly is composed of a fusion protein mentioned above and a hydrophobic degradable polyester or its derivative through hydrophobic interaction.

The third purpose of the present disclosure is to provide a preparation method for the nano-assembly which comprises the following steps:
(1) mix the fusion protein with water or an aqueous solution to obtain an aqueous phase; mix the hydrophobic degradable polyester and its derivatives with an organic solvent to obtain an oil phase;
(2) prepare the water phase and oil phase in step (1) into an oil-in-water emulsion,
(3) separate and purify the emulsion to obtain a nano-assembly.

The fourth purpose of the present disclosure is to provide an application of the nano-assembly in preparing the platform or system for antibody delivery.

The fifth purpose of the present disclosure is to provide an antibody delivery platform or system, comprising the above-mentioned nano-assembly and at least one antibody required for delivery.

The sixth purpose of the present disclosure is to provide an application of the above-mentioned nano-assembly as an immunotherapy drug.

The seventh purpose of the present disclosure is to provide an application of the fusion protein in the above-mentioned nano-assembly.

Compared with the prior art, the present disclosure has the following beneficial effects:

The present disclosure, based on extensive research and development in the early stage, prepares nanoparticles (assemblies) for delivering at least one monoclonal antibody by selecting fusion proteins of hydrophobic degradable polyester or its derivatives and specific proteins with hydrophobic domains. Hydrophobically degradable polyester or its derivatives are wound and assembled with the hydrophobic domain of the fusion protein through hydrophobic interactions, exhibiting excellent stability. The specific antibodies delivered by the nano-assembly of the protein Fc receptor fusion protein can quickly, efficiently, and controllably bind to one or more types of therapeutic monoclonal antibodies through simple physical mixing. It can maintain a complete structure during the long circulation process in the body, thus facilitating the “multivalence” and “multi-specificity” of antibodies, making the long-term development of this multi antibody delivery system possible for clinical application. The preparation method of the multi antibody delivery system only involving physical mixing of albumin-based nanoparticles with various antibodies in this present disclosure is simple, and can effectively enhance the killing effect on tumor cells without affecting the activity of the multi antibody under this delivery system or platform.

The present disclosure creatively applies the constructed nano-assembly platform to the preparation of immunotherapeutic drugs or therapeutic drugs for tumors, autoimmune diseases, or inflammation for the first time, and will have broad application prospects.

### BRIEF DESCRIPTION OF DRAWINGS

FIG. 1 shows the construction process of plasmid pPICZαA-mFcγRI-MSA.

FIG. 2 shows the PCR identification of the Yeast vector-target gene.

FIG. 3 shows the PCR identification of yeast recombinants.

FIG. 4 shows the plasmid map of pcDNA3.1(+)-hFcγRI-HS.

FIG. 5 shows SDS-PAGE and Western Blot analysis of purified mFcγRI-MSA.

FIG. 6 shows Western Blot analysis of hFcγRI-HSA.

FIG. 7 shows schematic diagram for preparing nano-adaptors.

FIG. 8 shows the particle size of the nano-adaptor NP_{mFcγRI-MS} at a concentration of 5 mg/mL.

FIG. 9 shows the scanning electron microscope images of the nano-adaptor NP_{mFcγRI-MS}.

FIG. 10 shows the image of serum stability of the nano-adaptor NP_{mFcγRI-MS}.

FIGS. 11A and 11B show combined efficiency chart of the nano-adaptor NP_{mFcγRI-MS} through ELISA determination. FIG. 11A the particle size of the nanoparticles of fusion protein mFcγRI-MSA-polylactic acid in PBS; FIG. 11B the particle size of the nanoparticles of fusion protein mFcγRI-MSA-polylactic acid in cell culture medium.

FIG. 12 shows the efficiency of nano-adaptor binding to therapeutic monoclonal antibodies over time.

FIG. 13 shows the expression of PD-L1 and PD-1 in B16-F10 melanoma cells and CD8±T cells stimulated in vitro.

FIG. 14 shows the combination of NP_{mFcγRI-MSA@ceD-1+0D-L1} and B16-F10 melanoma cells. (A) Time dependent curve of extracellular fluorescence intensity; (B) CLSM image of combination of B16-F10 cells and imNA_{αPD-1 & αPD-L1} at a measuring scale of 5 μM; (C) Flow histogram of fluorescence intensity changes with time before and after quenching of trypan blue: Trypan blue can quench extracellular fluorescence, so the fluorescence that can be detected by flow cytometry after quenching is considered as intracellular fluorescence. FITC fluorescence was labeled on NP.

FIG. 15 shows the combination of NP_{mFcγRI-MSA@αPD-1+αPD-L1} and CD8⁺T cells.

FIG. 16 shows the laser confocal observation of the interaction between tumor cells and CD8±T cells mediated by bispecific nano aptamers.

FIG. 17 shows the activity of B16-F10-luc melanoma cells measured by Luciferase method.

FIG. 18 shows the curve of bispecific nano antibody inhibiting the growth of breast cancer in situ.

FIG. 19 shows the graph of the body weight changes of mice treated with bispecific nano antibodies.

FIG. 20 shows the curve of trispecific antibody nano-adaptor inhibiting the growth of breast cancer in situ.

FIG. 21 shows the survival curve of trispecific antibody nano-adaptor inhibiting the growth of breast cancer in situ.

### DESCRIPTION OF EMBODIMENTS

In the following embodiments of the present disclosure, the experimental methods without specific conditions are usually in accordance with conventional conditions or in accordance with the conditions recommended by the manufacturers. Various common chemical reagents used in the examples are all commercially available products.

Unless otherwise defined, all technical and scientific terms used in the present disclosure are the same as commonly understood by those skilled in the art. The terms used in the specification of the present disclosure are only for the purpose of describing specific embodiments, but not used to limit the present disclosure.

The terms “including” and “having” and any variations thereof in the present disclosure are intended to cover non-exclusive inclusions. For example, a process, method, device, product, or equipment which includes a series of steps is not limited to the listed steps or modules, but optionally includes steps that are not listed, or optionally other steps which are includes inherently in those processes, method, product, or equipment.

The “plurality” mentioned in the present disclosure means two or more. “And/or” describes the association relationship of the associated objects, indicating that there can be three types of relationships, for example, A and/or B, which can mean: A exists alone, A and B exist at the same time, and B exists alone. The character “/” generally indicates that the associated objects before and after are in an “or” relationship.

Homology: In the theory of biological phylogeny, if two or more structures share the same ancestor, they are called homology.

Affinity of antibodies refers to the binding strength between the antigen-binding cluster of an antibody and the antigen-determined cluster of an antigen, or the binding force between an antibody and an antigenic epitope or antigen-determined cluster. Essentially, it is a non-covalent force that includes attraction to amino acids, hydrogen bonding, hydrophobic forces, etc.

An embodiment of the present disclosure relates to a fusion protein, comprising a protein with a hydrophobic region, a peptide junction, and a protein receptor; protein fusion receptors include Fc receptors.

Fc receptors are receptors that bind to Fc fragments of antibodies (IgG), including FcγRI, FcγRII and FcγRIII The Fc receptor of the present disclosure is a receptor that specifically binds to the Fc segment of the delivered antibody, preferably FcγRI. Further, the Fc receptor has the same or similar genetic origin as the delivered antibody, preferably mFcγRI (Rat Fcγ RI) or hFc γ RI (human Fc γ RI).

In some embodiments, protein receptors include Fc receptors of antibodies, and the Fc receptors of the antibodies include but are not limited to: Fcγ Receptors (FcγR). For example, mouse Fc receptor mFcγRI, Human Fc Receptor hFcγRI.

In some embodiments, the FcγRI of the present disclosure is the extracellular segment of natural proteins.

FcγRI is non covalently bound to the Fc domain of the delivered monoclonal antibody; the delivered antibody has affinity with the fusion protein.

In some embodiments, the protein has at least Fc receptors and serum albumin fragments that can bind to hydrophobic degradable polyester and its derivatives through hydrophobic interactions. In the present disclosure, it is albumin, i.e., serum albumin, and can be at least one kind from human serum albumin, bovine serum albumin, mouse serum albumin, mouse serum albumin, rat serum albumin, rabbit serum albumin, and chicken egg albumin.

The serum albumin is homologous to the Fc receptor.

In some preferred embodiments, the fusion protein comprises a full length or partial fragment of albumin and Fc receptor protein, or a protein that has been substituted, deleted, mutated, and/or added with one or more naturally occurring, non naturally occurring, or modified amino acids, but does not lose its corresponding function or role in the delivery antibody system. In some embodiments, the fusion protein is composed of mouse serum albumin MSA and mouse Fc receptor, or human serum albumin HSA and human Fc receptor; the sequence of the mouse serum albumin MSA is GENEBANK BC049971.1, with the signal peptide sequence and stop codon removed, as shown in SEQ ID No. 1; the sequence of mouse Fc receptor mFcγRI is GENEBANK NM_010186.5, with the sequence of signal peptide, transmembrane region and intracellular segment removed, as shown in SEQ ID No. 2; the sequence of human serum albumin HSA is GENEBANK HQ537426.1, with the sequence of signal peptide and stop codon removed, as shown in SEQ ID No. 3; the sequence of human Fc receptor hFcγRI is GENEBANK BC152383.1, with the sequence of signal peptide, transmembrane region and intracellular segment removed, as shown in SEQ ID No. 4.

Peptide connectors can be a sequence of connectors commonly used to connect peptides, which can connect two peptides and naturally fold them into the desired structure. Typically, they are short peptides with hydrophobicity and certain extensibility. In the present disclosure, the purpose is to separate the fused two proteins to alleviate their mutual interference. The peptide connector can be flexible. In some embodiments, flexible peptide connectors may be advantageous as they can connect two protein/peptide components while maintaining their respective activity and function. This type of peptide connector includes, but is not limited to, (GGGGS) n. In some embodiments, the peptide connector is [GlyGlyGlyGlySer]n, wherein n is an integer from 0 to 4, more preferably 1, 2, 3, 4. When n is zero, it means that the fusion protein can be directly connected from the serum albumin to the protein receptor.

In some embodiments, the fusion protein is sequentially composed of serum albumin, peptide connector, and protein receptor from the N-terminal to the C-terminal.

In some embodiments of the present disclosure, a preparation method for the fusion protein involves the following steps: (a) constructing a recombinant Pichia pastoris cell line; (b) the fusion protein was induced to express in its growth medium for 4 days, with an expression level of 30 mg/L; (c) purification of the protein expressed in step(b).

The polynucleotides encoding various proteins with hydrophobic domains, such as serum albumin and FcγRI, can be obtained by methods known in the art, such as PCR, RT-PCR, artificial synthesis, construction and screening of cDNA library, etc. The mRNA or cDNA used as PCR template and for construction of cDNA library can be derived from any tissue, cell, library, etc. containing corresponding mRNA or cDNA, such as human liver fetal cDNA library. It can also be obtained through artificial synthesis, where the host's preferred codon can be selected to improve the expression of the product. The polynucleotides encoding IL1ra can be obtained from a human liver fetal cDNA library using RT-PCR. The fusion of polynucleotides encoding serum albumin and polynucleotides encoding FcγRI can be obtained by using various methods known in the art, such as introducing restriction endonuclease recognition sites on both sides of the coding sequence through PCR, producing sticky ends through enzymatic digestion, and then connecting the sticky ends with DNA ligase, so as to obtain genes encoding fusion proteins; fusion gene fragments can also be obtained through overlay PCR. If necessary, polynucleotides can be introduced on both sides of the gene encoding the fusion protein of the present disclosure, and the introduced polynucleotides can have restriction endonuclease recognition sites. Nucleic acids containing coding fusion protein sequences can be cloned into various expression vectors using well-known methods in the art. The host that expresses the fusion protein can be yeast, mammalian cells, bacteria, animals, plants, etc. Fusion proteins or peptides can exist within the host cell or be secreted from the host, preferably secreted from the host. Signal peptide for secretion, preferably yeast α-factor signal peptide or natural serum albumin signal peptide, or the analog of these two signals peptide. The yeast α-factor signal peptide is more preferable, and the fusion protein expression level is higher when using this signal peptide. The fusion protein or polypeptide can also be expressed in yeast in a soluble form without signal peptide. The nucleic acid encoding the fusion protein can be inserted into the host chromosome or exist in the form of a free plasmid.

Common methods such as electroporation and preparation of receptive protoplast can be used to transform the needed nucleic acid into host cells. Successfully transformed cells, i.e. cells containing the DNA construction of the present disclosure, can be identified through well-known techniques such as collecting and splitting cells, extracting genomes, and then identifying them using PCR methods. Alternatively, proteins in cell culture supernatant or cell fragmentation fluid can be detected using anti serum albumin or anti antibodies.

The fusion protein of the present disclosure can be produced by cultivating hosts containing the DNA construction of the present disclosure, such as recombinant yeast, recombinant mammalian cells, recombinant bacteria, genetically modified animals and plants, etc. The specific cultivation method can be achieved through shaking bottles or bioreactors, preferably bioreactor for production. The culture medium should be able to provide the substances required for the growth of bacteria (or cells) and the expressions of the products. It should include nitrogen sources, carbon sources, pH buffering components, etc. The culture medium formula should generally be obtained through experiments based on different culture objects. Culture can be divided into two stages. The first stage is mainly used for the growth of bacteria (or cells), and the second stage is mainly used for expressing products.

By collecting cell culture medium through centrifugation and concentrating the volume of the culture medium with a tangential flow device, fusion proteins can be isolated and purified from cell cultures containing the DNA construction of the present disclosure using various protein separation methods. Such as ultrafiltration, liquid chromatography, and combinations of these technologies, among which, liquid chromatography can use gel exclusion, affinity chromatography, ion exchange chromatography, hydrophobic chromatography, reverse phase chromatography and other chromatography techniques.

In some embodiments, a nano-assembly for antibody delivery is involved, which is composed of the fusion protein and hydrophobic degradable polyester or its derivatives through hydrophobic interaction.

The hydrophobic degradable polyester and its derivatives are currently known biodegradable biomaterials, as well as new degradable biomaterials developed in the future that can bind to the hydrophobic regions of the protein portion in the fusion protein. The polyester is an aliphatic polyester or its derivative, or a polyethylene glycol modified aliphatic polyester or its derivative.

In some embodiments, the aliphatic polyester is at least one from polylactide, polyglycolide, poly (lactide co lactide), and poly (caprolactone); or the polyethylene glycol modified aliphatic polyester is at least one from polyethylene glycol modified poly (lactide), polyethylene glycol modified poly (lactide co lactide), and polyethylene glycol modified poly (caprolactone).

In some embodiments, the aliphatic polyester is polylactide; the polylactide is poly(L-lactide), poly(R-lactide), or a racemic polylactide; the end group of the polylactide is at least one from ester, carboxyl, and hydroxyl groups. Preferably, the end group of the poly(L-lactide) is an ester group, which has stronger hydrophobicity.

In some embodiments, the polylactide is a poly(L-lactide), and the end group of the poly(L-lactide) is an ester group.

In some embodiments, the molecular weight range of the poly(L-lactide) is 7200 Daltons to 110000 Daltons, preferably 137000 Daltons to 240000 Daltons.

In some embodiments, the nano-assembly is a nano particle with a particle size range from 80 nm to 200 nm, preferably ranging from 80 nm to 150 nm.

In some embodiments of the present disclosure, there is a preparation method for the nano-assembly mentioned above, comprising the following steps:
(1) mix the fusion protein with water or an aqueous solution to obtain an aqueous phase, mix the hydrophobic degradable polyester and its derivatives with an organic solvent to obtain an oil phase;
(2) prepare the aqueous phase and oil phase in step (1) into an oil-in-water emulsion;
(3) separate and purify the emulsion to obtain a nano-assembly.

This embodiment provides a nano-adaptor for regulating immune reactions, consisting of a polyester and a fusion protein with a hydrophobic domain. The hydrophobic domain of the fusion protein binds to the polyester through hydrophobic interactions; The fusion protein is at least one from the albumin Fc receptors.

Among them, FcγRI can non covalently bind to the Fc domain of the delivered specific antibody; the specific antibody delivered has the same species origin as the anti Fc segment antibody or anti Fc segment antibody fragment.

The specific antibody delivered by the present disclosure has the same species source as the FcγRI has such as when the delivered specific antibody selects humanized anti PD-1 antibody, human source FcγRI was selected.

In some embodiments, the preparation process of the above-mentioned nanoparticles does not include additional stabilizers.

In some embodiments, nanoparticles and free proteins can be separated by at least one method from centrifugation, tangential flow dialysis (dialysis under tangential shear force through a tangential flow device), and exclusion chromatography (based on the molecular weight of nanoparticles and free proteins).

In some embodiments, the method of preparing the aqueous and oil phases into an oil-in-water emulsion includes ultrasonic emulsification, high-pressure homogenization emulsification, or microfluidic control.

In some embodiments, the weight ratio of the polyester or its solution to the fusion protein is 1:0.1 to 1:30, preferably 1:5 to 1:25, preferably 1:5 to 1:15, and more preferably 1:7 to 1: 11.

The concentration of the fusion protein in the aqueous phase is 0.5 mg/mL to 20 mg/mL, preferably 5 mg/mL to 10 mg/mL; the concentration of the polyester in the oil phase is 0.5 mg/mL to 10 mg/mL, with a preferred range of 1 mg/mL to 5 mg/mL.

Preferably, the volume ratio of the aqueous phase to the oil phase is 1:1 to 10:1, preferably 5 to 10:1, and more preferably 8:1 to 10:1.

In some embodiments, the organic solvent is chloroform or dichloromethane or similar compounds.

In one embodiment, the present disclosure relates to the application of the the above-mentioned nano-assembly in a platform or system for preparing antibody delivery.

In one embodiment of the present disclosure, an antibody delivery platform or system includes the above-mentioned nano assemblies and antibodies.

In some embodiments, the antibody to be delivered has at least one antibody, preferably two or three; and/or the antibody includes at least one monoclonal antibody, or specific antibody or antigen binding part thereof, preferably including two or more monoclonal antibodies, multivalent antibodies, humanized antibody, chimeric antibodies, and genetically engineered antibodies.

The delivery amount of at least one antibody can be the same or different, for example, it can be 1-10:1-10, preferably 1-5:1-5.

The at least one monoclonal antibody is PD-1 and PD-L1. Preferably, the amount of PD-1 and PD-L1 is 1-10:1-10, preferably 1-5:1-5.

In one embodiment of the present disclosure, an application of the above-mentioned nano-assembly as an immunotherapeutic drug.

In some embodiments, the immunotherapeutic drugs are tumor immunotherapeutic drugs or autoimmune disease treatment drugs.

In some embodiments, the nano-assemblies in the present disclosure can be assembled from FDA approved polymer polyester and albumin fusion proteins, exhibiting excellent biocompatibility.

The fusion protein in the present disclosure binds to antibodies through receptor ligand specific recognition. The inventor found that this structure will not destroy the structure of antibodies, and antibodies will not interact with each other. It overcomes the defects of traditional chemical bond fixation, such as destroying the structure of antibody drugs, blocking their antibody recognition regions, significantly affecting the function of antibody drugs, high complexity and high difficulty. It provides a new and simple structural design for the development of combined antibody therapy.

In addition, the nano-assembly in the present disclosure can also expose the Fab segment of the antibody outward, thereby maximizing the preservation of the antibody's function.

In some embodiments, it has been proven through a large number of in vitro and in vivo pharmacological experiments that the nano-assembly combining with specific antibodies to obtain a monoclonal antibody delivery system NP_{mFcγR1@αPD-1+αPD-L1} has significant advantages over free monoclonal antibody combination therapy. As it can significantly promote the interaction between effector cells and target cells and enhance the anti-tumor ability mediated by T cells. In some embodiments, NP_{mFcγRI-MSA} efficiently combines with monoclonal antibodies to form bilayer antibody nanoparticles with multivalent, multi specific, and multifunctional properties. It can quickly combine with different therapeutic antibodies to adapt to the current strategy of personalized treatment under precise treatment in clinical practice, and has enormous clinical application potential.

PD-1 (Programmed Death Receptor 1), also known as CD279 (Differentiation Cluster 279), is an important class of immunosuppressive molecules. It regulates the immune system and promotes self-tolerance by downwardly regulating the response of the immune system to human cells and inhibiting the inflammatory activity of T cells, which can not only prevent autoimmune diseases, but also prevent the immune system from killing cancer cells.

In the present disclosure, various publicly available PD-1 antibodies, PD-L1 antibodies, and any PD-1 antibodies or PD-L1 antibodies that have been improved upon.

Polylactic acid, also known as polylactide, (C₃H₄O₂) n, is a new type of biodegradable material obtained from the polymerization of lactic acid as the main raw material.

The present disclosure will be further described in detail as below. The relevant sequences used in the following embodiments.

MSA
SEQ ID No. 1
aggggtgtgtttcgccgagaagcacacaagagtgagatcgcccatcggtataatgatttgggagaacaacatttcaaaggcctagtcctgat
tgccttttcccagtatctccagaaatgctcatacgatgagcatgccaaattagtgcaggaagtaacagactttgcaaagacgtgtgttgccgat
gagtctgccgccaactgtgacaaatcccttcacactctttttggagataagttgtgtgccattccaaacctccgtgaaaactatggtgaactgg
ctgactgctgtacaaaacaagagcccgaaagaaacgaatgtttcctgcaacacaaagatgacaaccccagcctgccaccatttgaaaggc
cagaggctgaggccatgtgcacctcctttaaggaaaacccaaccacctttatgggacactatttgcatgaagttgccagaagacatccttattt
ctatgccccagaacttctttactatgctgagcagtacaatgagattctgacccagtgttgtgcagaggctgacaaggaaagctgcctgacccc
gaagcttgatggtgtgaaggagaaagcattggtctcatctgtccgtcagagaatgaagtgctccagtatgcagaagtttggagagagagctt
ttaaagcatgggcagtagctcgtctgagccagacattccccaatgctgactttgcagaaatcaccaaattggcaacagacctgaccaaagtc
aacaaggagtgctgccatggtgacctgctggaatgcgcagatgacagggcggaacttgccaagtacatgtgtgaaaaccaggcgactatc
tccagcaaactgcagacttgctgcgataaaccactgttgaagaaagcccactgtcttagtgaggtggagcatgacaccatgcctgctgatct
gcctgccattgctgctgattttgttgaggaccaggaagtgtgcaagaactatgctgaggccaaggatgtcttcctgggcacgttcttgtatgaa
tattcaagaagacaccctgattactctgtatccctgttgctgagacttgctaagaaatatgaagccactctggaaaagtgctgcgctgaagcca
atcctcccgcatgctacggcacagtgcttgctgaatttcagcctcttgtagaagagcctaagaacttggtcaaaaccaactgtgatctttacga
gaagcttggagaatatggattccaaaatgccattctagttcgctacacccagaaagcacctcaggtgtcaaccccaactctcgtggaggctg
caagaaacctaggaagagtgggcaccaagtgttgtacacttcctgaagatcagagactgccttgtgtggaagactatctgtctgcaatcctg
aaccgtgtgtgtctgctgcatgagaagaccccagtgagtgagcatgttaccaagtgctgtagtggatccctggtggaaaggcggccatgctt
ctctgctctgacagttgatgaaacatatgtccccaaagagtttaaagctgagaccttcaccttccactctgatatctgcacacttccagagaag
gagaagcagattaagaaacaaacggctcttgctgagctggtgaagcacaagcccaaggctacagcggagcaactgaagactgtcatgga
tgactttgcacagttcctggatacatgttgcaaggctgctgacaaggacacctgcttctcgactgagggtccaaaccttgtcactagatgcaa
agacgccttagcc
mFcγRI
SEQ ID No. 2
Gaagtggttaatgccaccaaggctgtgatcaccttgcagcctccatgggtcagtattttccagaaggaaaatgtcactttatggtgtgagggg
cctcacctgcctggagacagttccacacaatggtttatcaacggaacagccgttcagatctccacgcctagttatagcatcccagaggccag
ttttcaggacagtggcgaatacaggtgtcagataggttcctcaatgccaagtgaccctgtgcagttgcaaatccacaatgattggctgctactc
caggcctcccgcagagtcctcacagaaggagaacccctggccttgaggtgtcacggatggaagaataaactggtgtacaatgtggttttct
atagaaatggaaaatcctttcagttttcttcagattcggaggtcgccattctgaaaaccaacctgagtcacagcggcatctaccactgctcagg
cacgggaagacaccgctacacatctgcaggagtgtccatcacggtgaaagagctgtttaccacgccagtgctgagagcatccgtgtcatct
cccttcccggaggggagtctggtcaccctgaactgtgagacgaatttgctcctgcagagacccggcttacagcttcacttctccttctacgtg
ggcagcaagatcctggagtacaggaacacatcctcagagtaccatatagcaagggcggaaagagaagatgctggattctactggtgtgag
gtagccacggaggacagcagtgtccttaagcgcagccctgagttggagctccaagtgcttggtccccagtcatcagctcct.
HSA
SEQ ID No. 3
gatgcacacaagagtgaggttgctcatcggtttaaagatttgggagaagaaaatttcaaagccttggtgttgattgcctttgctcagtatcttca
gcagtgtccatttgaagatcatgtaaaattagtgaatgaagtaactgaatttgcaaaaacatgtgtagctgatgagtcagctgaaaattgtgac
aaatcacttcataccctttttggagacaaattatgcacagttgcaactcttcgtgaaacctatggtgaaatggctgactgctgtgcaaaacaaga
acctgagagaaatgaatgcttcttgcaacacaaagatgacaacccaaacctcccccgattggtcagaccagaggttgatgtgatgtgcactg
cttttcatgacaatgaagagacatttttgaaaaaatacttatatgaaattgccagaagacatccttacttttatgccccggaactccttttctttg
ctaaaaggtataaagctgcttttacagaatgttgccaagctgctgataaagctgcctgcctgttgccaaagctcgatgaacttcgggatgaaggga
aggcttcgtctgccaaacagagactcaaatgtgccagtctccaaaaatttggagaaagagctttcaaagcatgggcagtggctcgcctgag
ccagagatttcccaaagctgagtttgcagaagtttccaagttagtgacagatcttaccaaagtccacacggaatgctgccatggagatctgct
tgaatgtgctgatgacagggcggaccttgccaagtatatctgtgaaaatcaggattcgatctccagtaaactgaaggaatgctgtgaaaaac
ctctgttggaaaaatcccactgcattgccgaagtggaaaatgatgagatgcctgctgacttgccttcattagctgctgattttgttgaaagtaag
gatgtttgcaaaaactatgctgaggcaaaggatgtcttcctgggcatgtttttgtatgaatatgcaagaaggcatcctgattactctgtcgtgctg
ctgctgagacttgccaagacatatgaaaccactctagagaagtgctgtgccgctgcagatcctcatgaatgctatgccaaagtgttcgatgaa
tttaaacctcttgtggaagagcctcagaatttaatcaaacaaaactgtgagctttttgagcagcttggagagtacaaattccagaatgcgctatt
agttcgttacaccaagaaagtaccccaagtgtcaactccaactcttgtagaggtctcaagaaacctaggaaaagtgggcagcaaatgttgta
aacatcctgaagcaaaaagaatgccctgtgcagaagactatctatccgtggtcctgaaccagttatgtgtgttgcatgagaaaacgccagta
agtgacagagtcacaaaatgctgcacagagtccttggtgaacaggcgaccatgcttttcagctctggaagtcgatgaaacatacgttcccaa
agagtttaatgctgaaacattcaccttccatgcagatatatgcacactttctgagaaggagagacaaatcaagaaacaaactgcacttgttga
gcttgtgaaacacaagcccaaggcaacaaaagagcaactgaaagctgttatggatgatttcgcagcttttgtagagaagtgctgcaaggct
gacgataaggagacctgctttgccgaggagggtaaaaaacttgttgctgcaagtcaagctgccttaggctta
hFcγRI
SEQ ID No. 4
Caagtggacaccacaaaggcagtgatcactttgcagcctccatgggtcagcgtgttccaagaggaaaccgtaaccttgcattgtgaggtgc
tccatctgcctgggagcagctctacacagtggtttctcaatggcacagccactcagacctcgacccccagctacagaatcacctctgccagt
gtcaatgacagtggtgaatacaggtgccagagaggtctctcagggcgaagtgaccccatacagctggaaatccacagaggctggctacta
ctgcaggtctccagcagagtcttcacggaaggagaacctctggccttgaggtgtcatgcgtggaaggataagctggtgtacaatgtgcttta
ctatcgaaatggcaaagcctttaagtttttccactggaattctaacctcaccattctgaaaaccaacataagtcacaatggcacctaccattgct
caggcatgggaaagcatcgctacacatcagcaggaatatctgtcactgtgaaagagctatttccagctccagtgctgaatgcatctgtgacat
ccccactcctggaggggaatctggtcaccctgagctgtgaaacaaagttgctcttgcagaggcctggtttgcagctttacttctccttctacat
gggcagcaagaccctgcgaggcaggaacacatcctctgaataccaaatactaactgctagaagagaagactctgggttatactggtgcga
ggctgccacagaggatggaaatgtccttaagcgcagccctgagttggagcttcaagtgcttggcctccagttaccaactcctgtctggtttca
t
primer:
MSA-F
SEQ ID NO. 5
ggtggtggtggttctgaagcacacaagagt
MSA-R
SEQ ID NO. 6
gactctagaggctaaggcgtctttgcatct
mFcγRI-F
SEQ ID NO. 7
gcctcgagaaaagagaagtggttaatgccaccaaggc
mFcγRI-R
SEQ ID NO. 8
acagaaccaccaccaccaggagctgatga.

Materials and sources used in the example:
Fusion protein mFcγRI-MSA: expressed by recombinant yeast and purified by AKTA protein purification instrument.
Fusion protein mFcγRI-GS4-MSA: expressed by recombinant yeast and purified by AKTA protein purification instrument.
Fusion protein hFcγRI-(GS4)₂-HSA: expressed by recombinant HEK293T cells and purified by AKTA protein purification instrument.
Poly (lactic acid) PLA137K, molecular weight is 137000 Da, ester terminated poly(L-lactide): purchased from Jinan Daigang Biotechnology Co., Ltd.
Dichloromethane: purchased from Guangzhou Chemical Reagent Factory.
Anhydrous ethanol: purchased from China National Pharmaceutical Group Chemical Reagent Co., Ltd.
IgG1 antibodies from mouse sources: purchased from Bio X Cell Company in the United States. Sheep anti-mouse IgG antibody GOLD: purchased from Sigma Aldrich Company in the United States.
Transmission electron microscope copper mesh: purchased from Hyde Entrepreneurship (Beijing) Biotechnology Co., Ltd.
Protein free blocking solution: purchased from Shanghai Shenggong Biotechnology Co., Ltd.
His tag anti body (HRP, mouse anti body): purchased from Beijing Yiqiao Shenzhou Biotechnology Co., Ltd.
CD64 antibody (mouse antibody): purchased from Thermo Fisher Company in the United States. Albumin antibody (mouse antibody): Purchased from Abcam Company in the United States.
ELISA colorant: purchased from Beijing Yiqiao Shenzhou Biotechnology Co., Ltd.
PD-L1 antigen: purchased from Beijing Yiqiao Shenzhou Biotechnology Co., Ltd.
Rat anti PD-L1 antibody: purchased from Bio X Cell Companyin the United States.
Sheep-anti-rat IgG antibody HRP: purchased from Beijing Yiqiao Shenzhou Biotechnology Co., Ltd.
Polystyrene board used in ELISA: purchased from Corning Company in the United States.

The experimental instruments and models used in the examples:
Ultrasonic cell crusher: VCX130, Sonics, USA.
Rotary evaporator: RV 10 digital V digital display, IKA, Germany.
Microchannel reactor: 1300 SERIES A2, Corning Corporation, USA.
Nanoparticle and Zeta potentiometers: Nano ZSE, Malvern, UK.
Desktop micro freezing centrifuge: Microfuge 20R, Beckman Coulter, USA.
Transmission electron microscope: Tabs L120C, Thermo Fisher Scientific, USA.
Microplate reader: BioTek Company in the United States.

### EXAMPLE 1: Cloning of MSA cDNA

The MSA (Mouse Serum Albumin) cDNA without signal peptide coding sequence was obtained from mouse liver fetal cDNA library by PCR. The primers MSA-F (SEQ ID NO. 5) and MSA-R (SEQ ID NO. 6) used were synthesized with an oligonucleotide synthesizer. An XbaI cleavage sites and protective bases were introduced in the downstream primers, and the underlined area was the endonuclease recognition sequence.

50 μL of PCR reaction system: 25 μL of 2×Mix, DNA template<200 ng, 1 μL of Primer MSA-F (10 pmol/μL), 1 μL of Primer MSA-R (10 pmol/μL) plus ddH₂O supplement to 50 μL and the reaction system can be scaled down or enlarged according to demand. After gentle mixing, PCR was performed under the conditions of thermal denaturation at 94° C. for 1 min, followed by denaturation at 94° C. for 30 s, annealing at 58° C. for 30 s, extension at 72° C. for 1.5 min, a total of 30 cycles; and extending at 72° C. for 5 minutes. The expected 1.6 kb band was detected and analyzed by 1% agarose gel. The gel was recovered and quantified.

### EXAMPLE 2 Cloning of mFcγRI cDNA

Sequence of mFcγRI cDNA without signal peptide was obtained by gene synthesis, the primers mFcγRT-F (SEQ ID NO. 7) and mFcγRI-R (SEQ ID NO. 8) were synthesized with an oligonucleotide synthesizer Xba I cleavage sites and protective bases were introduced in the downstream primers.

50 μL of PCR reaction system: 25 μL of 2×Mix, DNA template<200 ng, 1 μL of Primer mFcγRI-F (10 pmol/μL), 1 μL of Primer mFcγRI-R (10 pmol/μL) plus ddH₂O supplement to 50 μL, and the reaction system can be scaled down or enlarged according to demand. After gentle mixing, PCR was performed under the conditions of thermal denaturation at 94° C. for 1 min, followed by denaturation at 94° C. for 30 s, annealing at 57° C. for 30 s, extension at 72° C. for 1.5 min for a total of 30 cycles; and extension at 72° C. for 5 minutes. The expected 1.7 kb band was obtained detected and analyzed by 1% agarose gel. And the gel was recovered and quantified.

### EXAMPLE 3 Fusion of Target Genes by Overlap PCR

50 μL of PCR reaction system: 25 μL of 2× Mix, 1 μL of Primer mFcγRI F (10 pmol/μL), 1 μL of Primer MSA R (10 pmol/μL), plus ddH₂O supplement. After gentle mixing, PCR was performed under the conditions of thermal denaturation at 94° C. for 1 min, followed by denaturation at 94° C. for 30 s, extension at 66° C. (−0.5° C./cycle) for 1.5 min, a total of 17 cycles; and then denaturation at 94° C. for 30 s, annealing at 58° C. (−0.5° C./cycle) for 30 s, extension at 72° C. for 1.5 min, a total of 5 cycles; then extension at 72° C. for 5 minutes.

### EXAMPLE 4: Construction of Yeast expression Vector of Fusion Gene

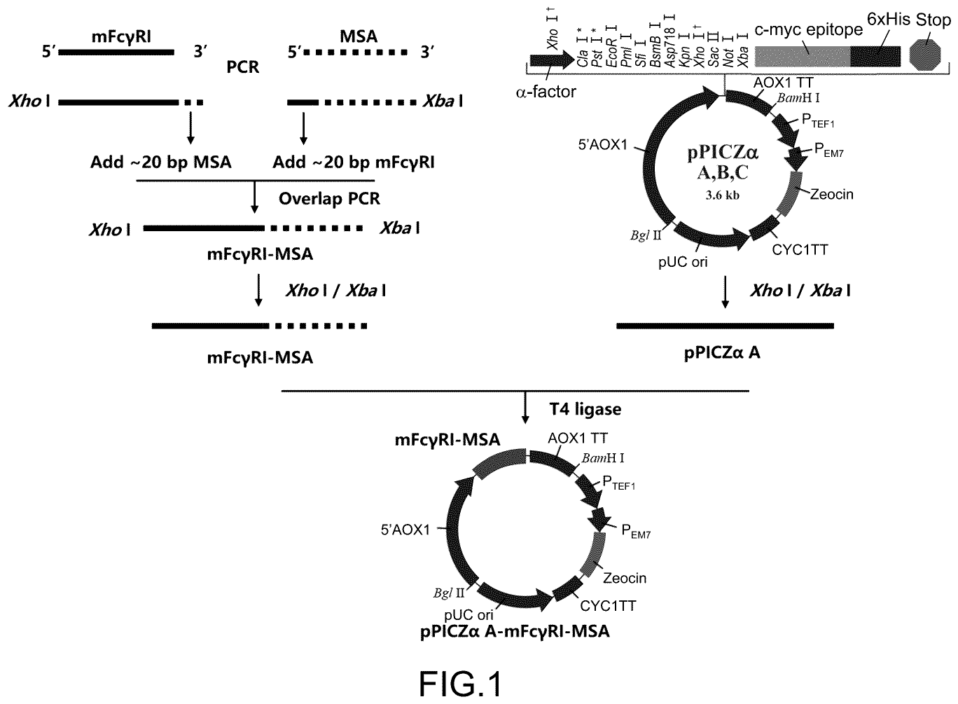

Xho I and Xba I were used to double enzyme digest mFcγRI-MSA fusion fragment and Yeast plasmid, and the 50 μL of reaction system of enzyme digestion was: 1 μg of mFcγRI-MSA Fragment and Yeast Plasmid, respectively, 1 μL each of Xho I and Xba I, 5 μL of CutSmart buffer, plus ddH₂O supplement to 50 μL. Enzymatic digestion was performed at 37° C. for more than 2 hours (preferably overnight without star activity), then inactivated by heat at 65° C. for 20 minutes. It was separated by agarose gel electrophoresis, and the gel with the target strip was recovered after cutting. Insertion fragments and the recovered plasmids was connected by T4 DNA ligase, 20 μL-linked reaction system: 2 μL of T4 Reaction Buffer, X μL of Vector DNA, Y μL of Insert DNA, Z μL of ddH₂O, 1 μL of T4 DNA Ligase. The system reacted at 25° C. for 20 minutes or stay overnight at 16° C., and the construction process of the plasmid vector is shown in FIG. 1.

### EXAMPLE 5 Transformation of Yeast Expression Vector in Escherichia coli

1 μL of plasmid (1 μg/μL) was diluted to 50 ng/μL with sterile water or TE buffer. E. Coli DH5a Competent Cells (100 μL) were thawed on ice before use, and added 1 μL of plasmid (<50 ng), placed in ice for 30 minutes, then placed at 42° C. for 45 seconds, immediately placed in ice for 1-2 minutes, avoiding shaking the centrifuge tube, then added antibiotic-free LB medium (pre-insulated at 37° C.) to 1 mL, mixed well and shook and cultured at 37° C. for 1 hour (200 rpm). An appropriate amount (<100 μL on a 100 mm plate)) was coated on a selected culture medium (low-salt LB medium containing 25 μg/mL Zeocin). The plate was placed on the front side for half an hour until the bacterial solution is absorbed, and incubated upside down at 37° C. overnight for 12-16 hours. The monoclonal was picked out, and cultured in low salt LB liquid medium containing 25 μg/mL Zeocin for amplification to extracted the plasmid.

### EXAMPLE 6 Colony PCR Identification of Escherichia coli

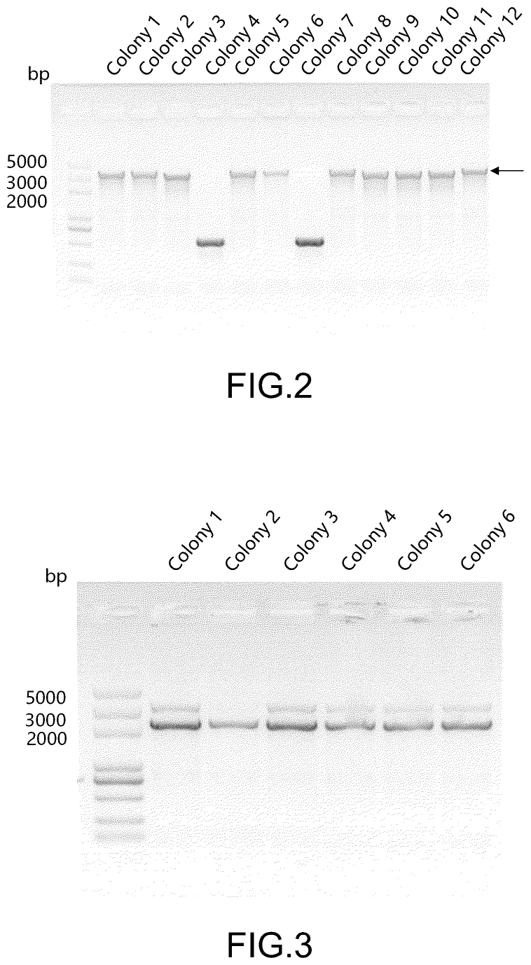

A single colony (colon, and numbered) was picked with a sterile tip, put into 20 uL of 0.1% Triton X-100, and stirred. The EP tube containing 20 uL of 0.1% Triton X-100 was boiled at 100° C. for 3 minutes, and centrifuged slightly for 1 minute, then 1 uL of supernatant was taken as a template. 20 μL of PCR reaction system is: 10 μL of 2×Mix, 1 μL of DNA template, 0.5 μL of Primer 5′AOX(10 pmol/μL), 0.5 μL of Primer 3′AOX (10 pmol/μL), 8 μL of ddH₂O. After gentle mixing, PCR was performed under the conditions of thermal denaturation at 94° C. for 1 min, followed by denaturation at 94° C. for 30 s, annealing at 54° C. for 30 s, extension at 72° C. for 1.5 min, a total of 30 cycles; and extension at 72° C. for 5 min. The expected 3.2 kb band was detected and analyzed by 1% agarose gel. The gel was recovered and quantified, seen in FIG. 2. The single colony was amplification cultured in LB liquid culture medium (containing antibiotics). After 18 hours of cultivation, 1 mL of bacterial solution was taken for sequencing.

### EXAMPLE 7 Chemical Transformation of Yeast

Plasmid DNA wan linearized and dephosphorylated, 50 μL of enzyme digestion reaction system is 5 μg of plasmid DNA, 5 μL of CutSmart Buffer (10X), 1 μL of Pmel, 1 μL of Quick CIP add ddH₂O to 50 μL. Enzyme digestion was performed at 37° C. for more than 2 hours in a PCR instrument, and stopped by heat inactivation at 65° C. for 20 minutes; Agarose gel electrophoresis was used to identify if the digestion was complete.

A tube of competent cells was thawed at room temperature and added 3 μL linearized DNA vector. 1 mL of solution II was added to the DNA/cell mixture and mixed through a vortex or light bomb centrifuge tube. The conversion mixture was incubated in a water bath or incubator at 30° C. for 1 hour, mixing with vortex or light bombing centrifuge tubes every 15 minutes for the conversion reactions. The cells were heat shocked in a hot block or water bath at 42° C. for 10 minutes, then were divided into two tubes (approximately 525 μL each) and added 1 mL of YPD culture medium into each tube. The cells were incubated at 30° C. for 1 hour to express the Zeocin resistance gene, then were pelleted by centrifugation at room temperature at 3000xg for 5 minutes, discard the supernatant. The cells of each tube were resuspended with 500 μL of solution III, and the two tubes of cells were combined into one tube. The cells were pelleted by centrifugation at room temperature at 3000×g for 5 minutes, discard the supernatant. The cells were resuspended with 100-150 μL of solution III. All transformants were screened on appropriate plates using a sterile applicator. The plates were incubated at 30° C. for 3 to 10 days, and each transformation should produce approximately 50 colonies. 6-10 Zeocin-resistant Pichia pastoris transformants 5 were selected and analyzed for the presence of inserts using PCR, see FIG. 3.

### EXAMPLE 8 Induced Expression of Recombinant Yeast with Mutt (Shake Flask Culture)

A single colony was selected and placed in a 250 mL shake flask containing 25 mL of BMGY culture medium, incubated at 28-30° C., 250-300 rpm until OD600=2-6 (16-18 hours). 1 mL of culture medium was taken and frozen; the remain was centrifuged at 1500-3000 g at room temperature for 5 minutes, and the bacterial was collected, resuspended with BMMY to adjust OD600 about 1.0 (about 100-200 mL), and start to induce expression; the obtained bacterial solution was placed in a 1 L shake flask, sealed it with double-layer gauze or coarse cotton cloth, and placed it on a shaker at 20-30° C. with a rotational speed of 250-300 rpm to continue growing; 100% methanol was added into the culture medium every 24 hours until the final concentration is 0.5-1.0%; 1 mL of bacterial liquid sample was taken at different time points, and placed it in a 1.5 mL EP tube, and centrifuged at the maximum speed for 2-3 minutes; the supernatant and bacteria were collected respectively to analyze the expression level of the target protein and the optimal harvesting time of the bacterial liquid. The time points are generally taken as 0, 6, 12, 24, 36, 48, 60, 72, 84, and 96 hours.

### EXAMPLE 9 Induced Expression of Recombinant Yeast with Mutt (Fermenters Culture)

Recombinant yeast with Mut+ was inoculated in 100 mL of YPD medium (10 g/L yeast extract, 20 g/L tryptone, 10 g/L glycerol), and cultured on a shaker at 30° C. and 280 rpm for 24 hours. The culture was inoculated into a 5-liter fermentation tank containing 2 L of basic salt culture medium, wherein the preparation method of the basic salt culture medium was as follows: 3.5 mL/L concentrated phosphoric acid, 0.15 g/L CaSO₄·2H₂O, 2.4 g/L K₂SO₄, 1.95 g/L MgSO₄·7H₂O, 0.65 g/L KOH, autoclaved at 121° C. for 30 minutes, and then added 40 mL/L glycerol (autoclave at 121° C. for 30 minutes separately), 1 mL/L PTM₁ (formula: 6.0 g/L CuSO₄·5H₂O, CoCl₂·6H₂O, 3.0 g/L MnSO₄·H₂O, 0.02 g/L H₃BO₃, 65.0 g/L FeSO₄·7H₂O, 0.2 g/L NaMoO₄·2H₂O, 20.0 g/L ZnSO₄·7H₂O, 0.1 g/L Kl, 5 mL/L concentrated sulfuric acid, 0.5 ml/L of 0.02% biotin, filtered and sterilized). The pH of the culture medium was adjusted to 5.0 with ammonia water before inoculation. The fermentation process was controlled at 25° C., and dissolved oxygen was always greater than 30% saturation. After the glycerol was depleted, glycerol (50% glycerol, containing 12 mL/L PTM₁) was added, and the culture continued until the density OD600 value was about 150, began to add methanol (analytical pure methanol, containing 12 mL/L PTM₁) to induce cultivation for 72 hours.

### EXAMPLE 10: Construction of pcDNA3.1 (+)-hFcγRI-HSA Vector

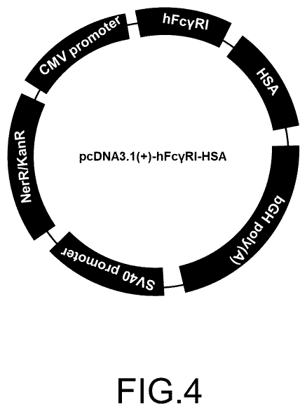

Double enzyme digestion of mFcγRI-MSA fusion fragment and Yeast plasmidwas perfomed, and the 50 μL of reaction system of enzyme digestion was: 1 μg of hFcγRI-HSA Fragment and pcDNA3.1(+), respectively, 1 μL each of Nhel and Xba I, 5 μL of CutSmart buffer, add ddH₂O supplement to 50 μL. Enzymatic digestion was performed at 37° C. for more than 2 hours (preferably overnight without star activity), then inactivated by heat at 65° C. for 20 minutes. It was separated by agarose gel electrophoresis, and the gel with the target strip was recovered after cutting the target strip. Insertion fragments and plasmids recovered was connected by T4 DNA ligase, 20 μL-linked reaction system“: 2 μL of” T4 Reaction Buffer, X μL of Vector DNA, Y μL of Insert DNA, Z μL of ddH₂O, 1 μL of T4 DNA Ligase. The system reacted at 25° C. for 20 minutes or stay overnight at 16° C., and the map of pcDNA3.1-hFcγRI-HSA is shown in FIG. 4.

### EXAMPLE 11 Transfection of pcDNA3.1(+)-hFcγRI-HSA into HEK293T Cells

20 μg plasmid was mixed with RPMI 1640 serum-free medium to 500 μL, and 60 μg PEI was mixed with RPMI 1640 serum-free medium to 500 μL. The plasmid mixture was added dropwise with F12-K/PEI mixture, mixed well, and incubated at room temperature for 20 minutes, with the EP tube being gently flicked. After incubation, the mixture was mixed with approximately 1×10 7 cells and cultured at 37° C. for 6-8 hours, then the medium was replaced with serum-free RPMI 1640 medium, and cultured for 72 hours, then the supernatant was harvested.

### EXAMPLE 12 Culture Medium Supernatant Concentrated Through Tangential Flow

1 L of bacterial solution cultivated for 96 hours was centrifuged at 5250×g for 5-10 minutes and the supernatant was collected; the bacterial solution was concentrated to 100 mL through tangential flow, then added another 500 mL of PBS, and concentrated again to 50 mL.

### EXAMPLE 13 Purification of Fusion Protein

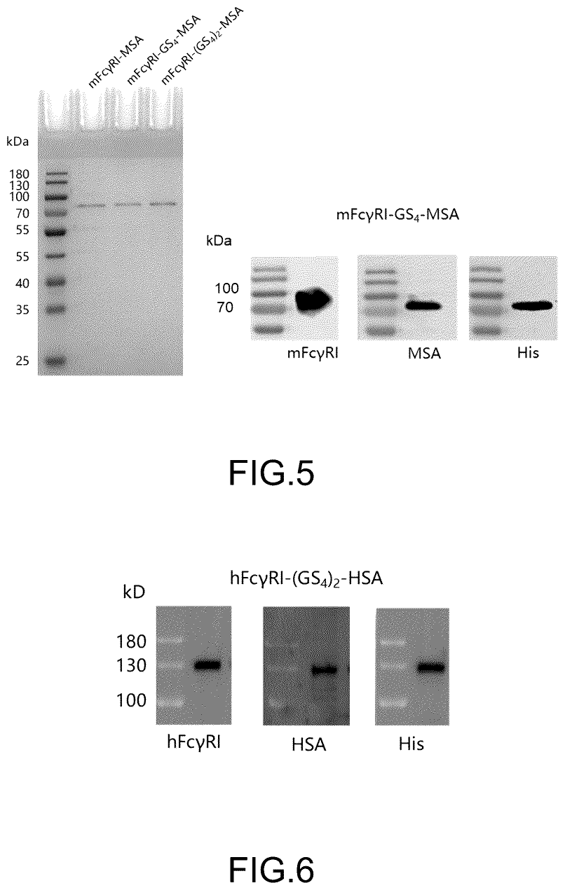

The nickel column was balanced with 5 column volumes of ddH₂O, and then balanced 10 column volumes with Native Binding Buffer. Sampled the concentrated culture medium, washed 10 column volumes with Native Wash Buffer, and then the protein was washed with Native Elution Buffer. Collected the fractions to obtain the purified fusion protein mFcγRI-MSA. The SDS-PAGE and Western Blot characterization results of mFcγRI-MSA are shown in FIG. 5, and the characterization results of hFcγRI-HSA are shown in FIG. 6.

### EXAMPLE 14 Preparation of Albumin Fusion Protein-Polylactic Acid Nanoparticles

(1) Ultrasonic emulsification method

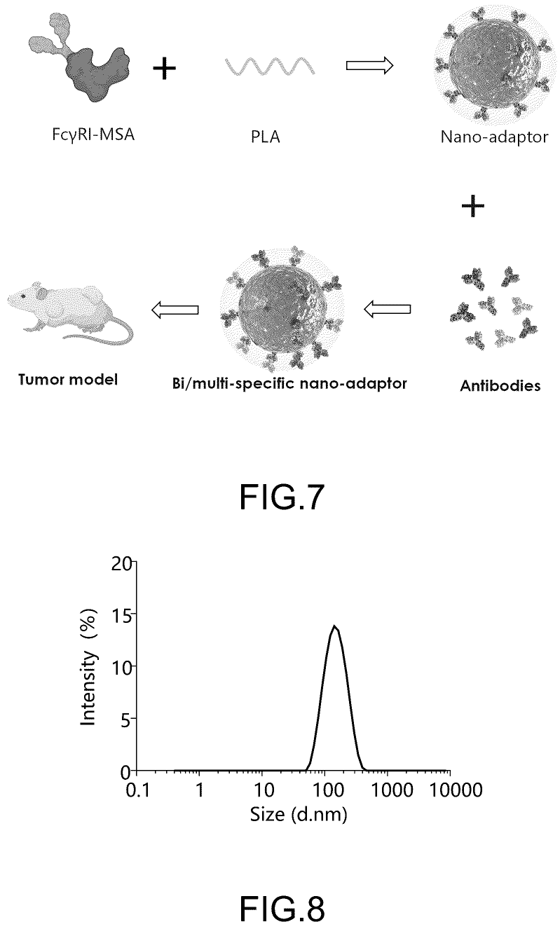

The purified fusion protein mFcγRI-MSA (concentration determined by Nanodrop One ultra-micro ultraviolet spectrophotometer) was prepared into a 5 mg/mL solution with ultrapure water, and a 5 mg/mL chloroform solution of polylactic acid (PLA₁₃₇ₖ) was prepared. Took 1 mL aqueous solution of 5 mg/mL fusion protein mFcγRI-MSA into a 15 mL centrifuge tube, added 100 μL of 5 mg/mL polylactic acid (PLA137k) chloroform solution (i.e. the mass ratio of fusion protein mFcγRI-MSA to PLA₁₃₇ₖ is 10:1). The mixture was emulsified in an ice water bath using an ultrasonic cell fragmentation device, the ultrasound power being 130 W, the amplitude being 50%, the ultrasound time being 1.5 minutes, and every 5 seconds ultrasound followed with 2 seconds pause (the interruption time was not included in the ultrasound time). After the ultrasonication, the emulsion was transferred to a 100 mL round bottom flask, and the residual emulsion in the centrifuge tube was washed out with ultrapure water, and the washing liquid was transferred into the 100 mL round bottom flask. The round bottom flask was rotated in the Rotary evaporator according to the vacuum degree of 200/100/50/30/20 mbar in sequence, maintaining for 10 minutes under each vacuum degree, wherein, under the vacuum degree of 30/20 mbar, the round bottom flask was immersed in a 32° C. water bath to fully remove the chloroform and evaporate a certain volume of water to concentrate the volume of the nanoparticle solution. After the rotary evaporation, the nanoparticles of fusion protein mFcγRI-MSA-polylactic were collected for later use, and the schematic diagram thereof is shown in FIG. 7. The nanoparticle preparation methods with different molecular weights and different types of polyester and fusion protein mFcγRI-MSA, and different ratios of polyester and fusion protein mFcγRT-MSA were referred to the above preparation method.

(2) Microfluidic technology

The purified fusion protein mFcγRT-MSA (concentration determined by Nanodrop One ultra-micro ultraviolet spectrophotometer) was prepared into a 5 mg/mL solution with ultrapure water, and a 2.5 mg/mL chloroform solution of polylactic acid was prepared. The second and third injection pumps of the microchannel reactor were select for the preparation of nanoparticles, wherein PLA137k chloroform solution was injected from the second injection pump; the aqueous solution of fusion protein mFcγRT-MSA was injected from the third injection pump. Before injection, the tubing was firstly rinsed with anhydrous ethanol at the maximum flow rate, and then the respective injection pipings were rinsed with the injected sample solvents (chloroform and water) at the maximum flow rate. After rinsing, the injection rate of PLA₁₃₇ₖ chloroform solution was set at 1.6 mL/min, and the injection rate of aqueous solution of fusion protein mFcγRI-MSA was set at 6.4 mL/min (i.e. the volume ratio of the aqueous phase to the organic phase is 4:1, and the mass ratio of fusion protein mFcγRI-MSA to PLA₁₃₇ₖ-chloroform is 8:1). When the emulsion produced at the sample outlet was uniform and stable the sample was collected into a 100/250 mL round bottom flask, then used a Rotary evaporator to rotate and evaporate sequentially according to the vacuum degree of 200/100/50/30/20 mbar, maintaining for 10 minutes under each vacuum degree, wherein, under the vacuum degree of 30/20 mbar, the round bottom flask was immersed in a 32° C. water bath to fully remove the chloroform and evaporate a certain volume of water to concentrate the volume of the nanoparticle solution. After rotary evaporation, the nanoparticles of fusion protein mFcγRI-MSA-polylactic acid were collected.

EXAMPLE 15 Purification Method of Nanoparticles of Fusion Protein mFcγRI-MSA-Polylactic Acid (Centrifugation Method)

The nanoparticles prepared in Example 12 were centrifugated at a low speed (3000 rpm, 5 minutes, at 4° C.) using a desktop micro freezing centrifuge to remove unassembled polylactic acid. The supernatant was transferred to a new EP tube for high-speed centrifugation (15000 rpm, 2 hours, 4° C.) to precipitate nanoparticles. Free protein in the supernatant was removed, and the lower layer of particles were resuspended with 1×PBS suspension for later use.

### EXAMPLE 16 Characterization of Particle Size of Nanoparticles of Fusion Protein mFcγRI-MSA-Polylactic Acid

Took 100 μL of purified and resuspended particle solution in Example 13 and placed it in the Zetasizer cells, and the hydration diameter and dispersity of the nanoparticles were measured by the nanoparticle size and Zeta potentiometer. The measured particle size was about 130-140 nm, and the distribution was uniform, and the corresponding particle size distribution diagram of the nanoparticle is shown in FIG. 8. The particle sizes of nanoparticles with different molecular weights, different types of polyesters and fusion protein mFcγRI-MSA, and different ratios of polyester to fusion protein mFcγRI-MSA are summarized as follows:

| | | Fusion protein: | Particle sizes of |
|---|---|---|---|
| No. | Types of polyester | polyester (w:w) | nanoparticles |
| 1 | PLA₇₂ₖ | 5:1 | 162.5 ± 5.47 |
| 2 | PLA₃₆ₖ | 5:1 | 154.8 ± 2.92 |
| 3 | PLA₂₄₀ₖ | 5:1 | 190.7 ± 6.89 |
| 4 | PLA₂₄₀ₖ | 10:1 | 147.2 ± 3.50 |
| 5 | PLGA(LA/GA = 50/50)₃₀ₖ | 10:1 | 120.5 ± 12.3 |

### EXAMPLE 17 Morphological Characterization of Nanoparticles of Fusion Protein mFcγRI-MSA-Polylactic Acid Through Transmission Electron Microscope

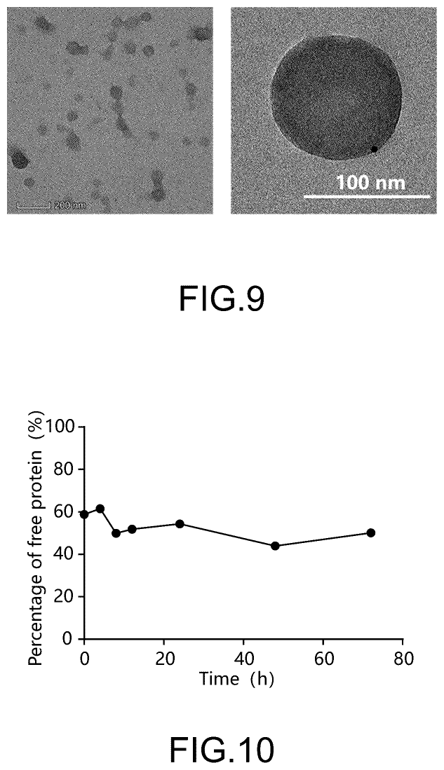

Took the purified and resuspended particle solution in Example 13, added mouse-derived IgG1 antibody and incubated overnight (8-10 hours) at 4° C. After the incubation, centrifuged (15000 rpm, 2 hours, 4° C.) to remove free and unbound antibodies, and the black particles bound to the antibodies precipitated from the lower layer were resuspended with 1×PBS. Then, added sheep anti-mouse IgG-antibody gold conjugate to the resuspended particle solution, incubated at 4° C. for 8 hours, centrifuged after incubation (15000 rpm, 20 minutes, 4° C.) to remove the unbound antibodies gold conjugate, and resuspended the red particles bounded with the antibodies gold conjugate in the lower layer with ultrapure water. The resuspended particle solution was diluted appropriately (the particle solution was diluted to an attenuator of 8 and a count rate of about 200 kcps through a nanoparticle degree and Zeta potentiometer), and took 2 μL and dropped it onto a transmission electron microscope (TEM) copper mesh, and allowed to air dry for 8 hours, and then observed under TEM. As shown in FIG. 9, nanoparticles of fusion protein mFcγRI-MSA-polylactic acid exhibited a spherical shape.

### EXAMPLE 18 Determination of Protein Assembly Rate and Protein Release Behavior of Nanoparticles of Fusion Protein mFcγRI-MSA-Polylactic Acid

Took the purified and resuspended particle solution in Example 13 and divided it into 7 equal parts, placed them in a shaker at 37° C., and took out a part at each time point (0, 4, 8, 12, 24, 48, 72 hours)and centrifuged it (15000 rpm, 2 hours and at 4° C.). After centrifugation the supernatant was collected and stored at −20° C. After the supernatant at all time points were collected, ELISA experiments were conducted to determine the fusion protein content in the supernatant at each time point.

ELISA method: The fusion protein mFcγRI-MSA was used as a standard, and the supernatant obtained at each time point was appropriately diluted as a sample. Plated the standard and samples (100 μL per well) and incubated at 4° C. overnight; washed with PBST after incubation to remove proteins that are not bound to the plate; Then the protein-free blocking solution was mixed with ultrapure water at 1:1, and added 200 μL to each well. After incubation at 37° C. for 1 hour, the plate was washed with PBST to remove residual blocking solution; and then, incubated with His-tag antibody (HRP) at 37° C. for 45 minutes, the plate was washed with PBST to remove unbound His-tag antibody (HRP), and then developed. During color development, mixed Solution A and Solution B at a ratio of 1:1, with 100 μL per well. After 8-10 minutes of dark color development, the plate was added 2 mol/L H₂SO₄ to terminate the color development, and the values of OD450 nm and OD630 nm was immediately detected by a Microplate reader.

Linear fitting on the linear region of the standard curve was performed, and the protein content in each supernatant sample was calculated accordingly so as to determine the protein assembly rate of nanoparticles of fusion protein mFcγRI-MSA-polylactic acid to be about 47%. As shown in FIG. 10, the nanoparticles exhibited good stability without significant protein release over a period of 72 hours.

### EXAMPLE 19 In Vitro Stability of Nanoparticles of Fusion Protein mFcγRI-MSA-Polylactic Acid

(1) Stability in PBS

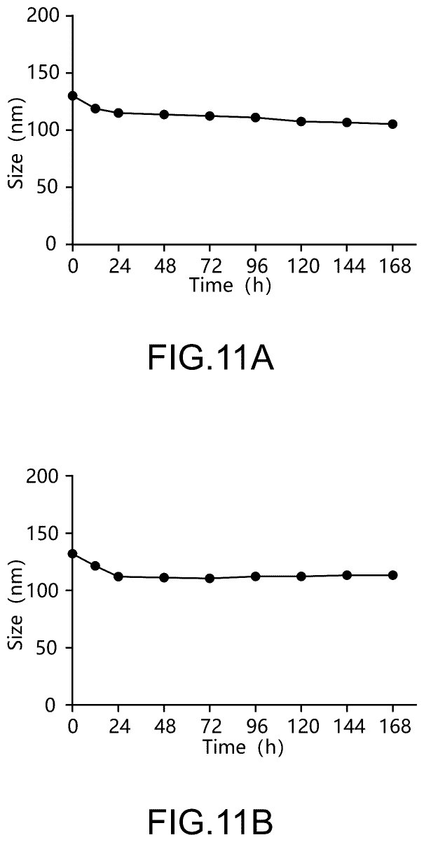

Took the purified and resuspended particle solution in Example 13 and divided it into 7 equal parts, placed them in a shaker at 37° C., and took out a part at each time point (0, 4, 8, 12, 24, 48, 72 hours) and measured the particle size by a nanoparticle size and Zeta potential meter. As shown in FIG. 11A, within 72 hours, the particle size of the nanoparticles of fusion protein mFcγRI-MSA-polylactic acid did not show significant changes, indicating that the fusion protein nanoparticles of the present disclosure have good stability in PBS.

(2) Stability in serum

The nanoparticles prepared in Example 12 were centrifugated at a low speed (3000 rpm, 5 minutes, 4° C.) using a desktop micro freezing centrifuge) to remove unassembled polylactic acid. The supernatant was transferred to a new EP tube for high-speed centrifugation (15000 rpm, 2 hours, 4° C.) to precipitate nanoparticles and removed free protein in the supernatant. The lower precipitate was resuspended with DMEM medium (adding 10% FBS), and then divided into 8 equal parts, placed them in a shaker at 37° C., and took out a part at each time point (0, 6, 18, 24, 32, 48, 72 and 96 hours) to measure the particle size by a nanoparticle size and Zeta potential meter. As shown in FIG. 11B, within 96 hours, the particle size of the nanoparticles of fusion protein mFcγRT-MSA-polylactic acid did not show significant changes, indicating that the fusion protein nanoparticles of the present disclosure also have good stability in cell culture medium.

### EXAMPLE 20, Antibody Binding Efficiency of Nanoparticles Fusion Protein mFcγRI-MSA-Polylactic Acid

The amount of αPD-L1 antibody was set consistent (10 μg), added different amounts of the purified and resuspended particle solution in Example 13 according to different mass ratios of particles and antibodies (250:1, 100:1, 50:1, 25:1, 10:1, 5:1, 2:1, 1:1), and then added PBS to make up the volume of each group of samples to 500 μL, and incubated overnight at 4° C. Groups of free antibodies (no particles) in the same volume setting were involved in this study as control. After incubation, all the samples were centrifuged at 15000 rpm for 2 hours. Took the supernatant, and measured the antibody concentration in the supernatant by ELISA.

ELISA method: αPD-L1 antibody was used as the standard, and the supernatant obtained at each time point was diluted 2000 times as a sample. The standard and samples were laid on a plate (100 μL per well) and incubated at 4° C. overnight. Washed with PBST after incubation to remove antigens that were not bound to the plate; then the protein-free blocking solution was mixed with ultrapure water at 1:1, and 200 μL were added to each well. After incubation at 37° C. for 1 hour, the plate was washed with PBST to remove residual blocking solution. Afterwards, the αPD-L1 antibody standard and diluted supernatant samples were incubated as primary antibodies at 37° C. (100 μL per well) for 1 hour, washed the plate with PBST to remove unbound primary antibody. Then sheep-anti-rat IgG antibody HRP was added and incubated at 37° C. for 45 minutes, washed with PBST to remove unbound sheep-anti-rat IgG antibody HRP, and then color development was performed. During color development, Solution A and Solution B were mixed at a ratio of 1:1, 100 μL per well. After 8-10 minutes of color development in the dark, the plate was added 2 mol/L H₂SO₄ to terminate the color development, and the values of OD450 nm and OD630 nm were immediately detected by a Microplate reader.

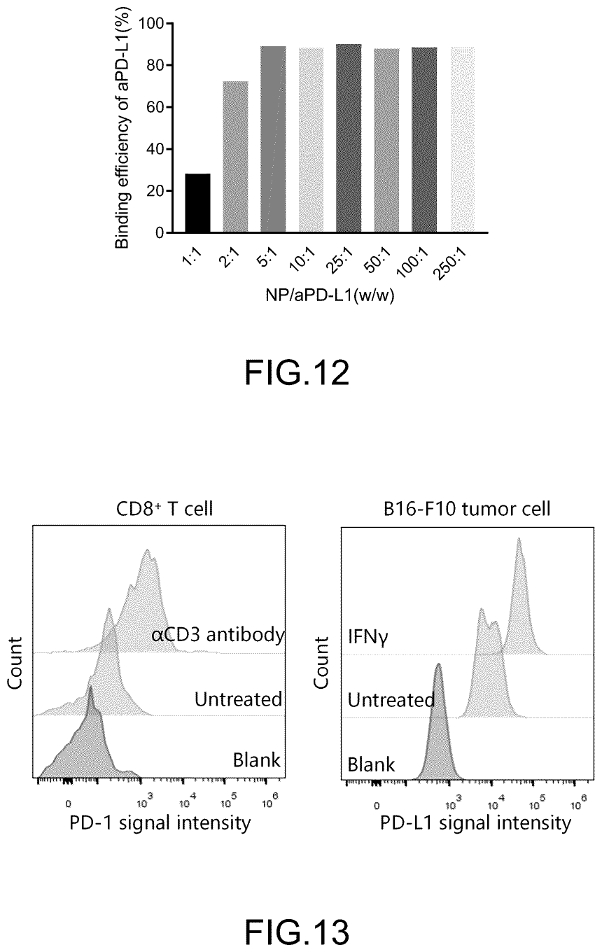

Linear fitting on the linear region of the standard curve was performed and the antibody content in each supernatant sample was calculated accordingly. The binding efficiency of antibodies under different particle-to-antibody mass ratios were calculated by using the antibody concentration measured by the free antibody (particle free) group as the original input. As shown in FIG. 12, the fusion protein nanoparticles of the present disclosure have excellent antibody binding ability.

### EXAMPLE 21 Binding of Serum Albumin Fusion Protein Bispecific Nanoantibodies to Tumor Cells and CD8±T Cells

Both the mouse melanoma cell line B16-F10 and the mouse orthotopic breast cancer cell line 4T1 were obtained from the American Type Culture Collection (ATCC). SPF grade C57BL/6 mice and female BALB/C mice, aged 5-6 weeks, were purchased from Hunan Slake Jingda Experimental Animal Co., Ltd. mice were raised in the Experimental Animal Center of South China University of Technology, and the animal experiment procedures followed the relevant regulations of the South China University of Technology's Regulations on the Management of Experimental Animals.

Rat anti-mouse PD-1 (CD279) antibody (αPD-1), and rat anti-mouse PD-L1 (B7-H1) antibody (αPDL1) were purchased from Bio X Cell, USA.

1. 10 ng/mL IFN-γ was used to stimulate and induce high expression of PD-L1 in B16-F10 cells (1.0×10 5 cells/well), and 5 μg/mL of αCD3ε was used to induce the activation of CD8⁺T cells from the spleen (5.0×10⁵ cells/well) to simulate the tumor microenvironment in vitro. Significant upregulation of PD-L1 expression in B16-F10 cells and upregulation of PD-1 expression in CD8±T cells were observed by flow cytometry (FIG. 13). The two kinds of cells activated by stimulation can be used as effective cells and target cells to simulate tumor microenvironment in vitro experiments.

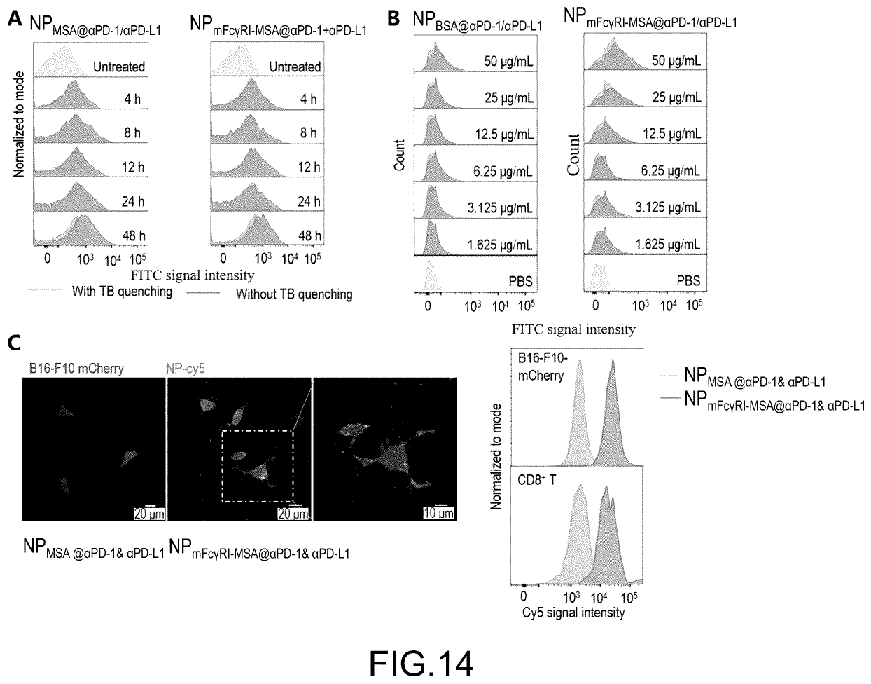

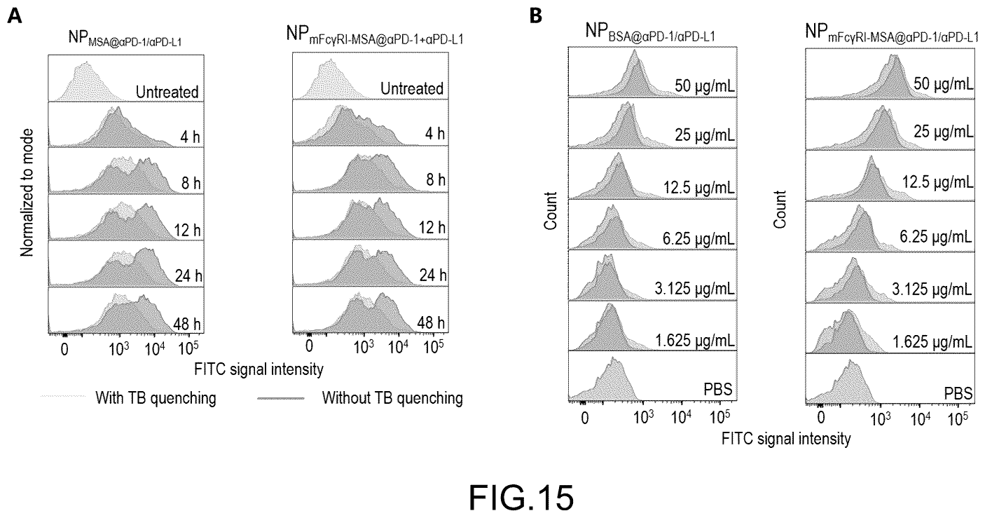

2. To evaluate the superiority of the nano antibody, we first investigated the ability of the serum albumin fusion protein nanoparticles bispecific antibody delivery platform to interact with cells. Fusion protein-polylactic acid complex NPmFcγRI-MSSA was prepared by ultrasonic emulsification method, using the fusion protein mFcγRI-MSA (5 mg/mL) in Example 11 and polylactic acid polymer PLLA 137k (5 mg/mL) as the basic components; Bispecific nano antibody NP_{mFcγRI-MSA@αPD-1&αPD-L1} was prepared by mixing NP CRI-MSA with anti-mouse PD-1 and PD-L1 antibodies (1:1 ratio) at a mass ratio of 25:1 (refer to Example 15). Using BSA (5 mg/mL) and polylactic acid polymer PLLA₁₃₇ₖ (5 mg/mL) as basic components, a fusion protein-polylactic acid complex NP_{BSA} was prepared by ultrasonic emulsification method; Bispecific nano antibody NP_{BSA@αPD-1 &αPD-L1} was prepared by mixing NP BSA with anti-mouse PD-1 and PD-L1 antibodies (1:1 ratio) at a mass ratio of 25:1. PD-L1 high B16-F10 cells (5.0×10⁴ cells/well and 1.0×10⁴ cells/dish) and PD-1high CD8±T cells (5.0×10⁴ cells/well and 1.0×10⁴ cells/dish) were co-incubated with FITC labeled NP_{BSA@αPD-1&αPD-L1} and NP_{mFcγRI-MSA@αPD-1&αPD-L1}, respectively (the concentration of αPD-1 & αPD-L1 was 20 μg/mL), and the target binding ability of NP_{mFcRI-MSA@αPD-1&αPD-L1} was evaluated by flow cytometry and confocal laser scanning microscopy (CLSM). As shown in FIG. 14A, the fluorescence intensity of B16-F10 cell and NP_{mFcγRI-MSA@αPD-1&αPD-L1} increased with the prolongation of incubation time; and we confirmed that the particles were on the surface of the cell membrane rather than entering the cell by quenching extracellular fluorescence with trypan blue. At the same time, we also set up NP_{BSA@αPD-1&αPD-L1} and NP_{mFcγRI-MSA@αPD-1} treatment groups with different concentrations of antibodies, and found through flow cytometry that when the antibody concentration was greater than 6.25 μg/mL, the mean fluorescence intensity (MFI) of NP_{mFcγRI-MSA@αPD-1 &αPD-L1} in B16-F10 cells and CD8⁺T cells increased with increasing concentration of antibody (FIG. 14B). The CLSM images also showed a large amount of NP_{mFcγm-MSA@αPD-1&αPD-L1} binding to the surface of B16-F10 cells (expressing mCherry fluorescent protein, proteins on NP was labeled with FITC) (FIG. 14C). For CD8±T cells, NP_{mFcγm-MSA@αPD-1&αPD-L1} also exhibited a time-dependent and dose-dependent combination, and almost no particles entered into CD8±T cells (FIG. 15). On the contrary, the control group NP_{BSA@αPD-1&αPD-L1} exhibited weak interactions with both types of cells (FIG. 14 and FIG. 15), indicating that the binding of NP_{mFcγRI-MSA@αPD-1&αPD-L1} to cells depended on the specific recognition and binding of the carried monoclonal antibody to the antigen. The above results proved that NP_{mFcγRI-MSA} can specifically bind co-inhibitory molecules a PD-1 & αPD-L1, while NP BSA can not specifically bind co-inhibitory molecules αPD-1 & αPD-L.

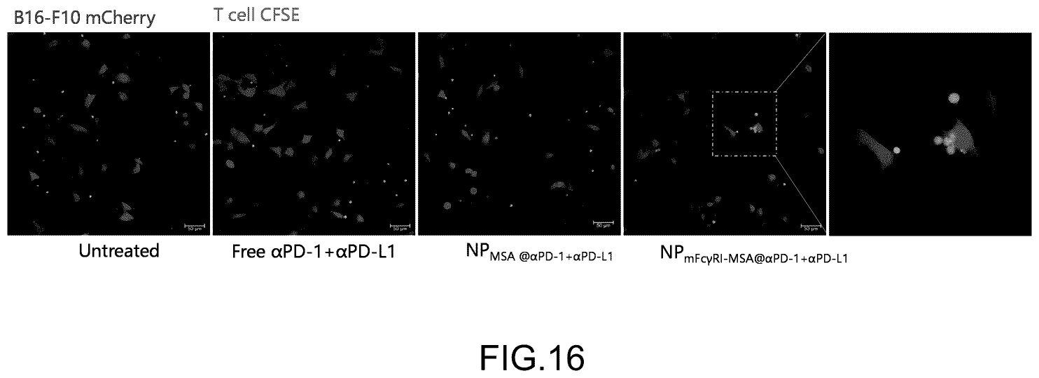

3. In order to explore the interaction between serum albumin fusion protein nanoparticles combined with therapeutic antibodies and cells, we selected a mouse melanoma cell line B16-F10. CD8±T cells isolated from the spleen were labeled with CFSE, and co-cultured with B16-F10 cells (expressing mCherry fluorescent protein). Four experimental groups were set: PBS control group, free αPD-1 and αPD-L1 hybrid group, NP BSA synchronously carrying αPD-1 and αPD-L1 group (NP_{BSA@αPD-1&αPD-L1}), and serum albumin fusion protein bispecific nano antibody group, i.e. NPmFcγRI-MSSA synchronously carrying αPD-1 and αPD-L1 group (NP_{mFcγm-MSA@αPD-1&αPD-L1}) (with [αPD-1], [αPD-L1] each 10 μg/mL). The cells in each group were treated accordingly, and after 4 hours of cultivation, unbound nanoparticles, antibodies, and CD8±T cells that did not interact with tumor cells were washed away. As shown in FIG. 16, compared with other groups, NP_{mFcγRI-MSA@αPD-1&αPD-L1} group exhibited more co-localization between CD8±T cells (green) and tumor cells (red), indicating that the nano antibody can promote the interaction between the two types of cells.

### EXAMPLE 22 In Vitro Cell Killing Assay of Serum Albumin Fusion Protein Bispecific Nanoantibodies

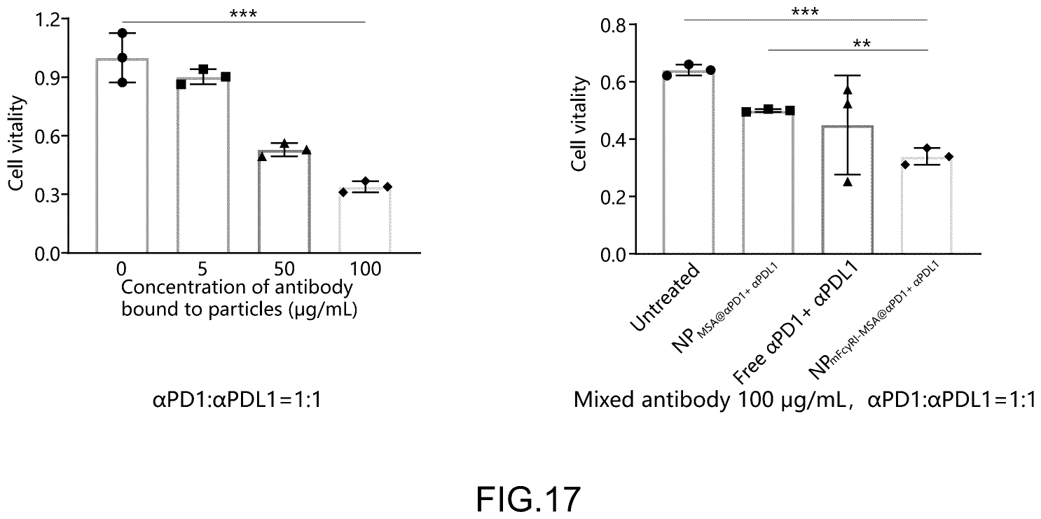

To understand whether NPmFcγRI-MSA@c(PD-1&c(PD-L1 could further activate CD8±T cells in vitro and promote their mediated cytotoxic effects, the sorted T cells was activated by αCD3ε antibody, and co-cultured with B16-F10 cells (expressing luciferase fluorescence). Six experimental groups were set: positive control group (adding 1% Triton), negative control group (adding equal volume of culture medium), PBS control group, free αPD-1 and a PD-L1 hybrid group, NP_{BSA} synchronously carrying αPD-1 and αPD-L1 group (NP_{BSA@αPD-1&αPD-L1}), and serum albumin fusion protein bispecific nano antibody group, i.e. NP_{mFcγRI-MSSA} synchronously carrying αPD-1 and αPD-L1 group (NP_{mFcγm-MSA@αPD-1&αPD-L1}), plus NP_{mFcγRI-MSA@αPD-1&αPD-L1} antibody treatment groups with different concentration groups. The cells in each group were treated accordingly and cultured for 24 hours in a 5% CO 2 environment at 37° C. Added 150 μg/mL of fluorescein, and immediately detected the chemiluminescence using a multifunctional microplate reader. Cell viability was calculated according to the formula: T cell viability (%)=[(experimental group OD value−positive group OD value)/(negative group OD value−positive group OD value)]. As shown in FIG. 17, more intracellular fluorescence in tumor cells was detected in the experimental group involved in NP_{mFcγRI-MSA@αPD-1&αPD-L1}, indicating a more effective tumor killing effect; and as the concentration of particles increased, the concentration of multivalent antibodies increased correspondingly, and the killing effect of T cells on tumor cells were effectively enhanced.

### EXAMPLE 23 Animal Level Anti-Tumor Therapy Experiment

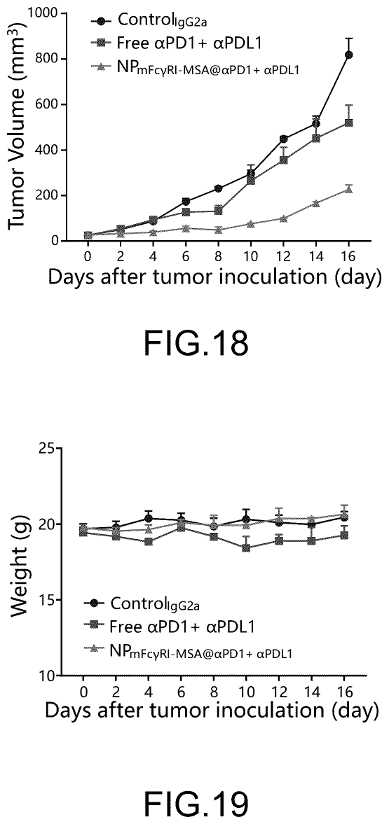

Fifteen BALB/C mice implanted with 4T1 orthotopic breast cancer were randomly divided into three groups with five mice in each group: PBS group (tail vein injection with 200 μL/mouse of PBS), Free αPD-1&αPD-L1 group (with 100 μg/mouse αPD-1& αPD-L1), NP_{mFcγRI-MSA@αPD-1&αPD-L1} group (with 2 mg/mouse of mFcγRI-MSA and 100 μg/mouse of αPD-1& αPD-L); administered once every three days, a total of three times. During the whole treatment process, the mice were weighed every 2 days and the tumor size was measured with a vernier caliper. Tumor volume was calculated using the following formula: volume (mm 3)=0.5×length×width. As shown in FIG. 18, the tumors in the PBS control group and the free antibody group grew rapidly, and the tumor growth in the NP_{mFcγRI-MSA@αPD-1&αPD-L1} group was significantly inhibited, which was due to the ability of the bispecific nano antibody to carry antibody drugs into the body and enhanced the interaction between tumor cells and T cells. As shown in FIG. 19, there was no significant change in the body weight of the mice in each group during the entire treatment process, indicating that the components of each group were not severely toxic to the survival of the mice.

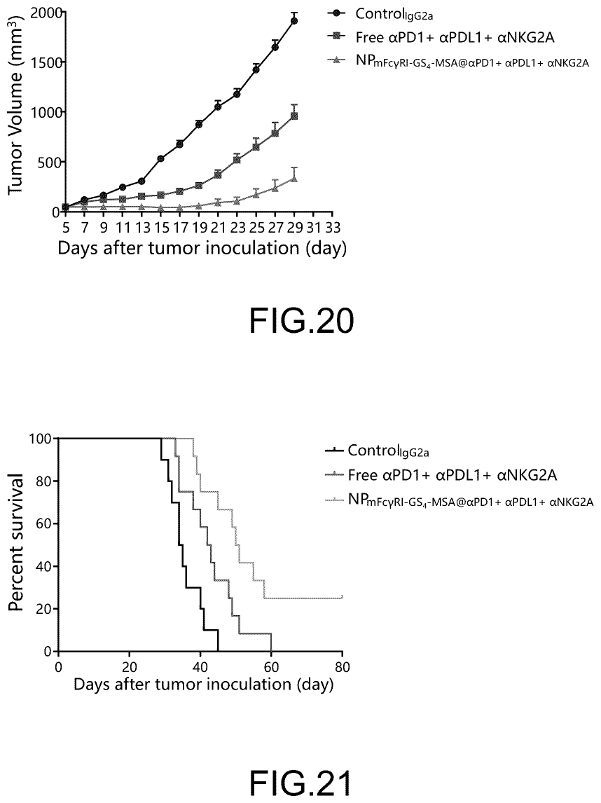

Thirty-six BALB/c mice implanted with 4T1 orthotopic breast cancer were randomly divided into 3 groups with 12 mice in each group: tail vein injection of 200 μL of PBS, αPD—1 &αPD-L1 &αNKG2A (100 μg/antibody/mouse, physically mixing of multiple antibodies; Free αPD-1&αPD-L1&αNKG2A group), NP_{mFcγRI-MSA@αPD-1&αPD-L1&αNKG2A} (3 mg/mouse of mFcγRI-GS4-MSA, 100 μg/antibody/mouse of αPD-1&αPD-L1&αNKG2A, nano-assembly (nanoparticles) and antibody mixture physically mixed; NP_{mFcγRI-GS4-MSA@αPD-1&αPD-L1&αNKG2A} group). The particle preparation method refers to Example 14, and administered once every three days, a total of two times. During the whole treatment process, the mice were weighed every two days and tumor size was measured with a vernier caliper, and the tumor volume was calculated according to the following formula: volume (mm 3)=0.5×length×width. As shown in FIG. 20, the tumors in the PBS control group and the free antibody group grew rapidly, and the tumor growth in the NP_{mFcγRI-GS4-MSA@αPD-1&αPD-L1&αNKG2A} group was significantly inhibited, which was because the antibody delivery system of NP_{mFcγRI-GS4-MSA@αPD-1&αPD-L1&αNKG2A} could carry antibody drugs into the body and enhanced the interaction between tumor cells, T cells and NK cells. As shown in FIG. 21, NP_{mFcγRI-GS4-MSA@αPD-1&αPD-L1&αNKG2A} can effectively prolong the survival of tumor-bearing mice.

The technical features of the examples above can be combined arbitrarily. To simplify description, all possible combinations of the technical features of the examples above are not described. However, as long as there is no contradiction in the combination of these technical features, they should be considered to be the scope of the specification.

The examples above merely express several implementations of the present disclosure. The descriptions of the examples are relatively specific and detailed, but may not therefore be construed as the limitation on the patent scope of the present disclosure. It should be noted that a person of ordinary skill in the art may further make several variations and improvements without departing from the concept of the present disclosure. These variations and improvements all fall within the protection scope of the present disclosure. Therefore, the patent protection scope of the present disclosure shall be defined by the appended claims.

What is claimed is:

1. A fusion protein for delivering at least one antibody, including serum albumin and a protein receptor, wherein the serum albumin is connected to the protein receptor directly or through a peptide connector; the protein receptor is an Fc receptor; the Fc receptor is a receptor that specifically binds to the Fc segment of the delivered antibody, and the Fc receptor has the same or similar genetic origin as the delivered antibody.

2. The fusion protein according to claim 1, wherein the Fc receptor is FcγRT.

3. The fusion protein according to claim 1, wherein the serum albumin is at least one of mammalian serum albumin, human serum albumin, bovine serum albumin, mouse serum albumin, mouse serum albumin, rat serum albumin, rabbit serum albumin, chicken egg albumin; and/or the serum albumin is homologous to the Fc receptor.

4. The fusion protein according to claim 1, wherein the Fc receptor is non-covalently bound to the Fc domain of the delivered antibody; and/or the delivered antibody has an affinity for the fusion protein.

5. The fusion protein according to claim 1, wherein the fusion protein comprises a full length or partial fragment of serum albumin and Fc receptor protein, or the above-mentioned protein that has been substituted, deleted, mutated, and/or added with one or more naturally occurring, non naturally occurring, or modified amino acids.

6. The fusion protein according to claim 1, wherein the fusion protein is sequentially composed of serum albumin, peptide connector, and protein receptor from the N-terminal to the C-terminal.

7. The fusion protein according to claim 1, wherein the fusion protein is the residue of the peptide connector can be [GlyGlyGlyGlySer]n, wherein n is an integer of 1-4.

8. A nano-assembly for delivering at least one antibody, being composed of a fusion protein according to claim 1 and a hydrophobic degradable polyester or its derivative through hydrophobic interaction.

9. The nano-assembly according to claim 8, wherein the hydrophobic degradable polyester or its derivative is a polyester, preferably an aliphatic polyester or its derivative, or a polyethylene glycol modified aliphatic polyester or its derivative.

10. The nano-assembly according to claim 9, wherein the aliphatic polyester is at least one of polylactide, polyglycolide, poly (lactide co lactide), and poly(caprolactone).

11. The nano-assembly according to claim 10, wherein the aliphatic polyester is polylactide; the polylactide is poly(L-lactide), poly(R-lactide), or a racemic polylactide; the end group of the polylactide is at least one of ester, carboxyl, and hydroxyl groups.

12. The nano-assembly according to claim 11, wherein the polylactide is a poly(L-lactide), and the end group of the poly(L-lactide) is an ester group.

13. The nano-assembly according to claim 12, wherein the molecular weight range of the poly(L-lactide) is 7200 Daltons to 110000 Daltons.

14. The nano-assembly according to claim 8, wherein the nano-assembly is a nano particle with a particle size range of 80 nm to 200 nm.

15. A preparation method of the nano-assembly according to claim 8, comprising the following steps:
(1) Mix the fusion protein according to claim 1 with water or an aqueous solution to obtain an aqueous phase with a concentration of 0.5 mg/mL to 20 mg/mL;
mix the hydrophobic degradable polyester and its derivatives with an organic solvent, at a concentration of 0.5 mg/mL to 10 mg/mL, to obtain an oil phase;
(2) Prepare the aqueous phase and oil phase in step (1) into an oil-in-water emulsion, the volume ratio of the aqueous phase to the oil phase is 1:1 to 10:1;
(3) Separate and purify the emulsion to obtain a nano-assembly.

16. An antibody delivery platform or system, comprising a nano-assembly according to claim 8, and at least one antibody to be delivered.

17. The antibody delivery platform or system according to claim 16, wherein the antibody to be delivered has at least one antibody; and/or the antibody includes at least one monoclonal antibody, or specific antibody or antigen-binding part thereof.

18. The antibody delivery platform or system according to claim 16, wherein the antibody includes two or more monoclonal antibodies, multivalent antibodies, humanized antibodies, chimeric antibodies, and genetically engineered antibodies.

19. A preparation method of an antibody delivery platform or system according to claim 16, wherein the antibody delivery platform or system is obtained by physically mixing the nano-assembly and at least one antibody to be delivered.
